SEQUENCE LISTING (13 sequences)

SEQ ID NO: 1 - DNA, 153 bp, Artificial
  single stranded targeting oligonucleotide MuKOFRT
ggacattggattattcgggatctgatggattagtgtgtaggctggagctgcttcgaagttcctatactttctagagaataggaacttcggaataggaactaaggaggatattcatatgtttgaagcgcgaaagctaaagttttcgcatttatc

SEQ ID NO: 2 - DNA, 24 bp, Artificial
  D5 primer
gatctgatcggattagatttggtg

SEQ ID NO: 3 - DNA, 24 bp, Artificial
  D6 primer
atgatgctagatgcattacctgaa

SEQ ID NO: 4 - DNA, 24 bp, Artificial
  E5 primer
tttgtaaccgacctgtatcagaaa

SEQ ID NO: 5 - DNA, 21 bp, Artificial
  E6 primer
agcatcaagaggatccatcag

SEQ ID NO: 6 - DNA, 24 bp, Artificial
  E11 primer
gcacaattattcagacaaagcact

SEQ ID NO: 7 - DNA, 24 bp, Artificial
  E12 primer
atcgttatctcgtgataccactca

SEQ ID NO: 8 - DNA, 20 bp, Artificial
  D7 primer
gattcagcaactggacgagg

SEQ ID NO: 9 - DNA, 24 bp, Artificial
  D8 primer
agtaaaaacagtccttttggatcg

SEQ ID NO: 10 - DNA, 24 bp, Artificial
  D9 primer
gcactgcaattaataaaaccaaaa

SEQ ID NO: 11 - DNA, 24 bp, Artificial
  D10 primer
acttatgctccataattctgaccg

SEQ ID NO: 12 - DNA, 90 bp, Artificial
  primer MuKO-R
tgaagcggcgcacgaaaaacgcgaaagcgtttcacgataaatgcgaaaactttagctttcgcgcttcaaacatatgaatatcctccttac

SEQ ID NO: 13 - DNA, 91 bp, Artificial
  MuKO-L primer
tgtattgattcacttgaagtacgaaaaaaaccgggaggacattggattattcgggatctgatgggattagtgtgtaggctggagctgcttc

(19) United States
(12) Patent Application Publication
(10) Pub. No.: US 2004/0214306 A1
(43) Pub. Date: Oct. 28, 2004

(54) Rapid growing microorganisms for biotechnology applications

(76) Inventors: Fredric R. Bloom, Germantown, MD (US); James Pfau, Oceanside, CA (US); Harry Yim, Vista, CA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/762,492

(22) Filed: Jan. 23, 2004

Related U.S. Application Data
(60) Provisional application No. 60/473,140, filed on May 27, 2003.
(60) Provisional application No. 60/441,742, filed on Jan. 23, 2003.

(51) Int. Cl. C12N 1/21

(52) U.S. Cl. 435/252.33

(57) ABSTRACT

The present invention provides novel rapid growing microorganisms and methods for their use in cloning or subcloning nucleic acid molecules. The rapid growing microorganisms of the present invention form colonies more rapidly than microorganisms typically used in molecular biology and thus provide a significant improvement in in vitro cloning methods used extensively in molecular biology. The rapid growing microorganisms of the invention preferably do not contain detectable levels of bacteriophage genetic material from at least one bacteriophage or in the alternative are resistant to infection by one or more bacteriophage types. The invention also relates to kits and compositions used in the methods of the invention.

### CROSS-REFERENCE TO RELATED APPLICATIONS

This application claims the benefit of U.S. Provisional Patent Appl. No. 60/441,742, Filed Jan. 23, 2003, and 60/473,140, Filed May 27, 2003, which are specifically incorporated herein by reference.

### BACKGROUND OF THE INVENTION

1. Field of the Invention

The present application relates to the field of biotechnology and, in particular, to the fields of cloning and protein expression.

2. Related Art

The fundamental process that sustains the ongoing biotechnology revolution is the cloning of DNA molecules for their further analysis or use. Cloning of DNA molecules has been practiced in the art for many years. A typical cloning protocol will involve identifying a desired DNA molecule, preparing a population of recombinant vectors by ligating the DNA molecule with a vector in a mixture of DNA molecule, vector and an appropriate ligase enzyme, transforming the population of recombinant vectors into a competent microorganism, growing the microorganism for some period of time sufficient to permit the formation of colonies, selecting colonies of microorganisms that potentially contain the desired DNA molecule correctly ligated in the vector, growing a sufficient quantity of each selected colony from which to isolate the recombinant vector, analyzing the isolated vector to ensure that the vector contains the desired DNA molecule and then growing a sufficient quantity of the microorganism that contains the correct recombinant vector to perform whatever subsequent manipulations are required. For details of various cloning procedures the reader may consult Sambrook, et al. 1989, Molecular Cloning: A Laboratory Manual 2^{nd} Ed. Cold Spring Harbor Laboratory Press, Cold Spring Harbor, N.Y., specifically incorporated herein by reference.

The typical cloning protocol outlined above thus includes at least three steps that involve growing of a microorganism. Since these growing steps generally require 12-16 hours and are usually performed as overnight incubations, the rate limiting steps for experiments involving cloning of a DNA fragment are the steps requiring growth of a microorganism. Although there are many variations on the basic practice of cloning, virtually all cloning methods require the insertion of the DNA molecule of interest into a microorganism and growth of the microorganism and, therefore, the speed of virtually every cloning methodology is limited by the rate of growth of the microorganism used for cloning.

For most cloning applications, the microorganism of choice is Escherichia coli (E. coli). Although numerous strains of E. coli are known, most cloning applications use one or another derivative of E. coli K-12. These derivatives suffer from the slow growth rate discussed above. Other known strains of E. coli, such as E. coli W (i.e., ATCC9637), have a rapid growth rate when compared to E. coli K-12; however, wild type strains of E. coli W and other rapid growing strains are not suitable for biotechnology applications for several reasons. First, the genetics of the organism have not been determined to the level of detail required by cloning applications. Thus, those skilled in the art would not know whether the genome of a microorganism contained the appropriate modifications of a number of genes that would make the microorganism suitable for biotechnology applications. For example, microorganisms are generally recA⁺ which leads to the formation of plasmid multimers and makes the microorganism less suitable for applications that involve the isolation of plasmid. Microorganisms typically contain numerous protease genes and may degrade overexpressed proteins thereby decreasing the yield of a desired protein product. Microorganisms typically contain a lac operon that does not permit alpha complementation and, therefore, the identification of recombinant vectors is more difficult. Further, many microorganisms contain endogenous plasmids that complicate the plasmid isolation steps necessary for cloning applications. In addition, microorganisms might contain genes coding for nucleases that could cause the degradation of exogenous plasmids. Finally, many microorganisms contain viruses; for instance, many bacterial strains are lysogenic for bacteriophage. Bacteriophage infection can interfere with plasmid isolation and purification from bacteria.

For a large number of biotechnology applications, a crucial step in the development of the application involves cloning one or more fragments of DNA. Given the central role of cloning in the development of the biotechnology industry, there has long existed in the art a need for reagents that speed the process of cloning. In particular, there exists a need in the art for microorganisms that have a desirable genotype and a rapid growth rate and can be employed to speed the cloning process. The present invention meets this long felt need.

### BRIEF SUMMARY OF THE INVENTION

The present invention provides microorganisms for biotechnology applications characterized by a rapid growth rate as compared to the microorganisms currently used for these applications. In particular, the present invention provides rapid growing microorganisms. The invention includes rapid growing microorganisms that lack endogenous plasmids and/or are free of bacteriophage infection. The invention also includes rapid growing microorganisms which are resistant to bacteriophage infection. The rapid growing microorganisms of the invention are therefore suitable for cloning applications. Because the microorganisms of the present invention form colonies faster than the microorganisms currently in use in cloning applications, the present invention provides an improvement in cloning desired nucleic acid molecules, allowing more rapid identification and isolation of recombinant vectors and clones of interest.

The present invention thus provides a method of cloning that employs a rapid growing microorganism. The method entails constructing a population of recombinant vectors, transforming competent microorganisms capable of rapid growth with the population of recombinant vectors, selecting a transformed microorganism containing one or more recombinant vectors of interest and/or isolating one or more recombinant vectors of interest from the transformed microorganism. The rapid growing microorganism may be, e.g., a rapid growing bacterium that lacks endogenous plasmids and/or is free of bacteriophage infection and/or is resistant or immune from bacteriophage infection. In one embodiment, the rapid growing microorganism is of the genus Escherichia. In another embodiment, the rapid growing microorganism is an E. coli. In a further embodiment, the rapid growing microorganism is an E. coli strain W.

In other embodiments, the rapid growing microorganism is selected from the group consisting of BRL3781, BRL3784 and recA⁻ derivatives thereof. The cloning methods of the present invention may optionally include a step of growing transformed microorganism at an elevated temperature to increase the growth rate of the microorganism, for example, at a temperature greater than 37° C. In a preferred embodiment, the transformed microorganisms may be grown at about 42° C.

The invention includes rapid growing bacteria or microorganisms that are free of bacteriophage infection and/or resistant to such infection. For example, the invention includes rapid growing bacteria that do not contain any bacteriophage genetic material, and/or have one or more genetic markers which prevent or inhibit infection with one or more bacteriophage types or have bacteriophage resistant phenotype. The invention also includes rapid growing bacteria or microorganisms that do not contain the genetic material of one or more specified bacteriophage types and/or have been modified or mutated to prevent or inhibit infection with one or more bacteriophage types. In one embodiment, the invention includes E. coli strain W that does not contain the genetic material of bacteriophage Wphi and/or does not contain the genetic material of bacteriophage Mu and/or is resistant to infection with T1 phage.

In certain embodiments, the rapid growing bacteria of the invention are resistant to infection by one or more bacteriophage type. The invention also includes methods for producing rapid growing bacteria that do not contain bacteriophage genetic material, and methods for producing rapid growing bacteria that are resistant to infection by one or more specified bacteriophage types. For example, the invention includes methods for producing rapid growing bacteria, e.g., E. coli strain W, that do not contain the genetic material of bacteriophage Wphi and/or that do not contain the genetic material of bacteriophage Mu.

The present invention provides a method of producing a protein or peptide which comprises constructing a recombinant vector containing a gene encoding a protein or peptide of interest, transforming the vector into a competent microorganism capable of rapid growth and culturing the transformed microorganism under conditions that cause the transformed microorganism to produce said peptide or protein. The rapid growing microorganism may be, e.g., a rapid growing bacterium that lacks endogenous plasmids and/or is free of bacteriophage infection. In a preferred embodiment, the rapid growing microorganism is of the genus Escherichia. In another preferred embodiment, the rapid growing microorganism is an E. coli. In another preferred embodiment, the rapid growing microorganism is an E. coli strain W. Other embodiments include a rapid growing microorganism deleted in the ion protease. In some preferred embodiments, the microorganism carries a gene encoding a T7 RNA polymerase (RNAP). In other preferred embodiments, the T7 RNAP gene is under the control of a salt inducible promoter, an arabinose inducible promoter, or an IPTG or lactose inducible promoter (e.g., in a lambda lysogen such as DE3).

The present invention also includes a method of producing a microorganism for cloning comprising the steps of obtaining a rapid growing microorganism containing endogenous plasmids and curing the microorganism of endogenous plasmids. In a preferred embodiment, the rapid growing microorganism is of the genus Escherichia. In another preferred embodiment, the rapid growing microorganism is an E. coli. In another preferred embodiment, the rapid growing microorganism is an E. coli strain W.

The present invention also includes a method of producing rapid growing bacteria for cloning comprising the steps of obtaining rapid growing bacteria that contain bacteriophage and curing the rapid growing bacteria of bacteriophage. In one embodiment, the rapid growing bacteria are E. coli. In a preferred embodiment, the rapid growing bacteria are E. coli strain W.

In a related aspect of the present invention, any desired modification or mutation may be made in the microorganisms of the present invention including, but not limited to, alteration of the genotype of the microorganism to a recA⁻ genotype such as recA1/recA13 or recA deletions, a lacZ⁻ genotype that allows alpha complementation such as lacX74 lacZAM15 or other lacZ deletion, a protease deficient genotype such as Δlon and/or ompT⁻, an endonuclease minus genotype such as endA1, a genotype suitable for M13 phage infection by including the F′ episome, a restriction negative, modification positive genotype such as hsdR17(r_{K}⁻, m_{K}⁺), a restriction negative, modification negative genotype such as hsdS20(r_{B}⁻, m_{B}⁻), a methylase deficient genotype such as mcrA and/or mcrB and/or mrr, a genotype containing suppressor mutations such as supE and/or supF. Other suitable modifications are known to those skilled in the art and such modifications are considered to be within the scope of the present invention.

The present invention provides a method of transforming a competent rapid growing microorganism comprising obtaining a recombinant vector and contacting a competent microorganism of the present invention with the recombinant vector under conditions which cause the rapid growing microorganism to take up the recombinant vector. The rapid growing microorganism may be, e.g., a rapid growing bacterium that lacks endogenous plasmids and/or is free of bacteriophage infection. In a preferred embodiment, the rapid growing microorganism is of the genus Escherichia. In another preferred embodiment, the rapid growing microorganism is an E. coli. In another preferred embodiment, the rapid growing microorganism is an E. coli strain W.

The methods of the present invention may optionally include the step of growing the transformed microorganism at elevated temperatures to increase the growth rate of the microorganism, for example, at a temperature greater than 37° C. In a preferred embodiment, the transformed microorganisms may be grown at about 42° C.

The present invention also includes rapid growing microorganisms that are unable to synthesize components of the cell membrane and therefore are unable to grow in media lacking the particular cell membrane component. Such microorganisms are useful, e.g., in cloning methods that involve the negative selection of cells that do not contain a desired exogenous plasmid. In a preferred embodiment, the rapid growing microorganism is a bacterium, e.g., an E. coli strain, that is unable to synthesize diaminopimelic acid. Also included in the present invention is a method for selecting for rapid growing bacteria, e.g., rapid growing bacteria that contain a plasmid of interest, said method comprising obtaining rapid growing bacteria that are unable to grow in media lacking diaminopimelic acid, transforming said rapid growing bacteria with a plasmid comprising a gene that restores the ability of said rapid growing bacteria to grow in the absence of diaminopimelic acid, and culturing the transformed rapid growing E. coli in medium lacking diaminopimelic acid.

The present invention also includes kits comprising a carrier or receptacle being compartmentalized to receive and hold therein at least one container, wherein the container contains rapid growing microorganisms. The kit optionally further comprises vectors suitable for cloning. In a preferred embodiment, the kits may contain a vector suitable for recombinational cloning. In a preferred embodiment, the rapid growing microorganisms may be competent. In some preferred embodiments, the rapid growing microorganisms may be chemically competent. In other preferred embodiments, the rapid growing microorganisms may be electrocompetent. In some preferred embodiments, the kits of the present invention may include one or more enzymes including, but not limited to, restriction enzymes, ligases, and/or polymerases. In other preferred embodiments, the kits of the present invention may include recombination proteins for recombinational cloning. The kits of the present invention may also comprise instructions or protocols for carrying out the methods of the present invention.

The present invention includes compositions comprising rapid growing microorganisms. In a preferred embodiment, the rapid growing microorganism may be a competent microorganism. In some preferred embodiments, the rapid growing microorganisms may be chemically competent. In other preferred embodiments, the rapid growing microorganisms may be electrocompetent. The compositions of the present invention may optionally comprise at least one component selected from buffers or buffering salts, one or more DNA fragments, one or more vectors, one or more recombinant vectors, one or more recombination proteins and one or more ligases. In a preferred embodiment, the compositions of the present invention may comprise a rapid growing microorganism in a glycerol solution. In other preferred embodiments, compositions of the present invention may comprise rapid growing microorganisms in a buffer. In preferred embodiments, the microorganisms of the present invention may be in a competence buffer. In other preferred embodiments, the compositions of the present invention may comprise a lyophilized rapid growing microorganism.

The present invention includes a method of making competent rapid growing microorganisms comprising the steps of obtaining a rapid growing microorganism, growing the rapid growing microorganism and treating the rapid growing microorganism to make it competent. In some embodiments of the present invention, treating the microorganisms may include the step of contacting the microorganisms with a solution containing calcium chloride. In other embodiments, treating may include the step of contacting the microorganisms with water. The rapid growing microorganism may be, e.g., a rapid growing bacterium that lacks endogenous plasmids and/or is free of bacteriophage infection. In a preferred embodiment, the rapid growing microorganism is of the genus Escherichia. In another preferred embodiment, the rapid growing microorganism is an E. coli. In another preferred embodiment, the rapid growing microorganism is an E. coli strain W.

### BRIEF DESCRIPTION OF THE DRAWING

FIG. 1 is a restriction map of the 5.5 kb plasmid of ATCC9637.

### DETAILED DESCRIPTION OF THE INVENTION

Definitions

In the description that follows, a number of terms used in recombinant DNA technology are utilized extensively. In order to provide a clear and more consistent understanding of the specification and claims, including the scope to be given such terms, the following definitions are provided.

Competent cells or competent microorganisms as used herein refers to cells or microorganisms having the ability to take up and establish exogenous DNA molecules. Competent cells include, but are not limited to, cells made competent by chemical means, i.e. chemically competent cells, as well as cells made competent for electroporation by suspension in a low ionic strength buffer, i.e. electrocompetent cells. The level of competence may vary depending on the need, the procedure used for preparing the competent microorganisms and the type of microorganism used. Various procedures to make competent microorganisms are available and well known to those skilled in the art. Examples of preferred levels of competence include 1 to 1×10¹², 1×10¹ to 1×10¹¹, 1×10² to 1×10¹⁰1, 1×10³ to 1×10⁹, 1×10⁴ to 1×10⁸, 1×10⁵ to 1×10⁷, transformants per μg? of nucleic acid (such as a reference plasmid including pUC 19, pUC 18 or other pUC derivative or pBR322). In another aspect, competence levels may include at least 1×10, at least 1×10², at least 1×10³, at least 1×10⁴, at least 1×10⁵, at least 1×10⁶, at least 1×10⁷, at least 1×10⁸, at least 1×10⁹, at least 1×10¹⁰, at least 1×10¹¹, and at least 1×10¹², transformants per μg? of nucleic acid.

Expression vector as used herein refers to a vector which is capable of enhancing the expression of a gene or portion of a gene which has been cloned into it, after transformation or transfection into a host cell. The cloned gene is usually placed under the control (i.e., operably linked to) certain control sequences such as promoter sequences. Such promoters include but are not limited to phage lambda P_{L} promoter, and the E. coli lac, trp and tac promoters, the T7 promoter and the baculovirus polyhedron promoter. Other suitable promoters will be known to the skilled artisan.

Gene as used herein refers to a sequence of nucleotides that is transcribed in a cell. The term includes sequences that code for proteins and/or peptides as well as other sequences that do not code for such proteins or peptides. Examples of genes that do not code for proteins include, but are not limited to, the genes for tRNA, rRNA and the like. A gene includes a promoter sequence to control the transcription of the gene. A gene may also contain other DNA sequence elements that regulate the amount or timing of transcription. Such sequences elements are seen to include, but are not limited to, enhancers and the like.

Cell or microorganism as used herein, and which terms may be used interchangeably with each other and with the terms “host cell” and “host cell strain,” includes microorganisms that can be genetically engineered. Both gram negative and gram positive prokaryotic cells may be used in accordance with the present invention. Typical prokaryotic host cells that may be used in accordance with the present invention include, but are not limited to, microorganisms such as those of the genus Escherichia sp. (particularly E. coli), Klebsiella sp., Streptomyces sp., Streptocococcus sp., Shigella sp., Staphylococcus sp., Erwinia sp., Klebsiella sp., Bacillus sp. (particularly B. cereus, B. subtilis, and B. megaterium), Serratia sp., Pseudomonas sp. (particularly P. aeruginosa and P. syringae) and Salmonella sp. (particularly S. typhi or S. typhimurium). It will be understood, of course, that there are many suitable strains and serotypes of each of the host cell species described herein, any and all of which may be used in accordance with the invention. Preferred as a host cell is E. coli, and particularly preferred are E. coli strains derived from E. coli W.

As used herein, a “derivative” of a specified microorganism is a progeny of the specified microorganism, a modified or mutated microorganism obtained or derived from the specified microorganism or its progeny, or other recipient microorganism that contains genetic material obtained directly or indirectly from the specified microorganism. Such a derivative microorganism may, for example, be formed by removing genetic material from a specified microorganism and subsequently introducing it into another microorganism (i.e., the progeny or other recipient microorganism) by any conventional methodology including, but not limited to, transformation, conjugation, electroporation, transduction and the like. A derivative may be formed by introducing one or more mutations or modifications into the genome or other genetic material (e.g. vectors, plasmids, extrachromosomal elements, etc.) of a microorganism. Such mutations or modifications may include one or more insertion mutations, deletion mutations and/or substitutions or various combinations thereof. The mutations or modifications may be insertions into the genome or other genetic material (e.g. vectors, plasmids, extrachromosomal elements, etc.) of the microorganism. Alternatively, the mutations may be deletions of one or more bases and/or nucleic acid sequences from the genome or other genetic material (e.g. vectors, plasmids, extrachromosomal elements, etc.) of the microorganism. In some instances, the mutations may be the alteration of one or more bases in the genome of the microorganism. Such modifications or mutations may also comprise substituting one or more nucleic acid bases and/or nucleic acid molecules for other nucleic acid molecules and/or bases. In addition, one microorganism is a derivative of a parent microorganism if it contains the genome of the parent microorganism but does not contain some or all of the same extrachromosomal nucleic acid molecules. Thus, a strain produced by curing some or all of the endogenous vectors from a parent strain is a derivative of the parent strain. Derivatives of a microorganism of the invention may also include those microorganisms obtained by the addition of one or more nucleic acid molecules into the microorganism of interest. Nucleic acid molecules which may be introduced into a microorganism will be recognized by one skilled in the art and may include, but is not limited to, vectors, plasmids, transposons, oligonucleotides, RNA, DNA, RNA/DNA hybrids, phage sequences, virus sequences, regardless of the form or conformation (e.g. linear, circular, supercoiled, single stranded, double stranded, single/double stranded hybrids and the like). Examples of mutations or other genetic alterations which may be incorporated into the microorganisms of the present invention include, but are not limited to, mutations or alterations that create: a recA⁻ genotype such as recA1/recA13 or recA deletions, a lacZ⁻ genotype that allows alpha complementation such as lacX74, lacZAM15 or other lacZ deletion, a protease deficient genotype such as Δlon and/or ompT⁻, an endonuclease minus genotype such as endA1, a genotype suitable for M13 phage infection by including the F′ episome, a restriction negative, modification positive genotype such as hsdR17(r_{K}⁻, m_{K}⁺), a restriction negative, modification negative genotype such as hsdS20(r_{B}⁻, m_{B}⁻), a methylase deficient genotype such as mcrA and/or mcrB and/or mrr, a genotype containing suppressor mutations such as supE and/or supF. Other suitable modifications are known to those skilled in the art and such modifications are considered to be within the scope of the present invention.

Insert or inserts as used herein refers to one or more desired nucleic acid segments.

Isolating as used herein means separating the desired material, component, or composition at least partially from other materials, contaminants, and the like which are not part of the material, component, or composition that has been isolated. For example, “isolating a recombinant vector” means treating a cell, tissue, organ or organism containing the recombinant vector in such a way as to remove at least some of the other nucleic acid molecules (e.g., large nucleic acid molecules) with which it may be associated in the cell, tissue, organ or organism. As one of ordinary skill will appreciate, however, a solution comprising an isolated recombinant vector may comprise one or more buffer salts and/or a solvents, e.g., water or an organic solvent such as acetone, ethanol, methanol, and the like, and yet the nucleic acid molecule may still be considered an “isolated” nucleic acid molecule with respect to its starting materials. In another example, to obtain an isolated microorganism, the microorganism of interest may be separated or purified at least partially from other microorganisms or components.

Plasmid as used herein refers to a stable extrachromosomal genetic element.

Promoter as used herein refers to a DNA sequence that controls the transcription from another DNA sequence. A promoter is generally described as the 5′-region of a gene and is customarily located proximal to the start codon. The transcription of an adjacent DNA segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

Rapid growing microorganism as used herein refers to a microorganism that grows more rapidly than a reference microorganism. Rapid growing microorganisms produce colonies of a defined size from individual cells faster than reference microorganisms. In general, a rapid growing microorganism will have an increased growth rate, such as a growth rate that is greater by 5%, 10%, 25%, 50%, 75%, 100%, 150%, 200%, 300%, 400%, 500%, 600%, 700%, 800%, 900%, 1000%, or greater than the growth rate of a reference microorganism. Greater increases in growth rate may be included depending upon the microorganisms compared. Reference microorganisms include microorganisms that are typically used for biotechnology applications. Exemplary reference microorganisms include E. coli K-12 derived strains. A preferred reference microorganism is E. coli MM294 (ATCC33625). Thus, a rapid growing microorganism, as used herein, includes a microorganism, e.g., a bacterium, having a growth rate that is at least 5%, 10%, 25%, 50%, 75%, 100%, 150%, 200%, 300%, 400%, 500%, 600%, 700%, 800%, 900%, 1000%, greater, than the growth rate of E. coli MM294. Other suitable reference microorganisms include E. coli strains DH5α and DH10B (Invitrogen Corporation, Carlsbad, Calif.). The invention also contemplates any microorganism which has an increased growth rate, such as a growth rate that is greater by 5%, 10%, 25%, 50%, 75%, 100%, 150%, 200%, 300%, 400%, 500%, 600%, 700%, 800%, 900%, 1000%, or greater, when compared to E. coli W, more particularly, the specified E. coli W strains described herein.

The term “rapid growing microorganism” includes rapid growing microorganisms that lack endogenous plasmids. The term “rapid growing microorganism” includes rapid growing microorganisms that do not contain any bacteriophage genetic material and rapid growing microorganisms that do not contain the genetic material of one or more particular bacteriophage types. The term “rapid growing microorganism” also includes rapid growing microorganisms that both lack endogenous plasmids and do not contain any bacteriophage genetic material or do not contain the genetic material of one or more particular bacteriophage types.

In determining whether a particular microorganism is a rapid growing microorganism, any method of determining growth rate known in the art can be used. For example, a rapid growing microorganism can be identified by comparing a putative rapid growing microorganism to a reference microorganism for the time required to grow a colony of 1 mm diameter on antibiotic containing LB plates after transformation with a plasmid conferring resistance to the antibiotic.

Rapid growing microorganisms of the present invention may also be identified by a comparison of the doubling time of a putative rapid growing microorganism to the doubling time of a reference microorganism. The rapid growing microorganisms of the present invention have a faster doubling time than reference microorganisms. Those skilled in the art are capable of determining the doubling time of a microorganism using standard techniques.

In determining whether a microorganism is a rapid growing microorganism, it is sometimes preferred that the reference microorganism and the putative rapid growing microorganism carry similar genetic markers or mutations. For example, a putative rapid growing microorganism that is recA⁻ should be compared to a recA⁻ reference strain. Those skilled in the art will appreciate that a recA⁻ microorganism may have a slower growth rate than a comparable recA⁺ microorganism.

Recombinant microorganism as used herein refers to any microorganism which contains a desired cloned gene in a recombinant vector, cloning vector or any DNA molecule. The term “recombinant microorganism” is also meant to include those host cells which have been genetically engineered to contain the desired gene on the host chromosome or genome.

Recombinant vector as used herein includes any vector containing a fragment of DNA that is not endogenous to the vector.

Vector as used herein refers to a nucleic acid molecule (preferably DNA) that provides a useful biological or biochemical property to an insert. Examples include plasmids, phages, viruses, autonomously replicating sequences (ARS), centromeres, transposons, and other sequences which are able to replicate or be replicated in vitro or in a host cell, or to convey a desired nucleic acid segment to a desired location within a host cell. A vector can have one or more restriction endonuclease recognition sites at which the sequences can be cut in a determinable fashion without loss of an essential biological function of the vector, and into which a nucleic acid fragment can be spliced in order to bring about its replication and cloning. Vectors can further provide primer sites, e.g., for PCR, transcriptional and/or translational initiation and/or regulation sites, recombinational signals, replicons, selectable markers, etc. Clearly, methods of inserting a desired nucleic acid fragment which do not require the use of homologous recombination, transpositions or restriction enzymes (such as, but not limited to, UDG cloning of PCR fragments (U.S. Pat. No. 5,334,575, entirely incorporated herein by reference), T:A cloning (e.g., U.S. Pat. Nos. 5,487,993 and 5,827,657), and the like) can also be applied to clone a fragment into a cloning vector to be used according to the present invention. The cloning vector can further contain one or more selectable markers suitable for use in the identification of cells transformed with the cloning vector.

The present invention may be used with vectors suitable for recombinational cloning as disclosed in U.S. Pat. Nos. 5,888,732 and 6,277,608 which are specifically incorporated herein by reference. Vectors for this purpose may comprise one or more engineered recombination sites. Vectors suitable for recombinational cloning may be linear or circular. When linear, a vector may include DNA segments separated by at least one recombination site. When circular, a vector may include DNA segments separated by at least two recombination sites. In one embodiment, a vector may comprise a first DNA segment and a second DNA segment wherein the first or the second may comprise a selectable marker. In other embodiments, a vector may comprise a first DNA segment and a second DNA segment, the first or the second segment comprising a toxic gene. In other embodiments, a vector may comprise a first DNA segment and a second DNA segment, the first or the second DNA segment comprising an inactive fragment of at least one selectable marker, wherein the fragment of the selectable marker is capable of reconstituting a functional selectable marker when recombined across the first or second recombination site with another inactive fragment of a selectable marker.

In accordance with the invention, any vector may be used. In particular, vectors known in the art and those commercially available (and variants or derivatives thereof) may be used in accordance with the invention. Such vectors may be obtained from, for example, Vector Laboratories Inc., Invitrogen, Promega, Novagen, NEB, Clontech, Boehringer Mannheim, Pharmacia, EpiCenter, OriGenes Technologies Inc., Stratagene, Perkin Elmer, Pharmingen and Research Genetics. Such vectors may be used for cloning or subcloning nucleic acid molecules of interest and therefore recombinant vectors containing inserts, nucleic acid fragments or genes may also be used in accordance with the invention. General classes of vectors of particular interest include prokaryotic and/or eukaryotic cloning vectors, expression vectors, fusion vectors, two-hybrid or reverse two-hybrid vectors, shuttle vectors for use in different hosts, mutagenesis vectors, recombinational cloning transcription vectors, vectors for receiving large inserts (yeast artificial chromosomes (YAC's), bacterial artificial chromosomes (BAC's) and P1 artificial chromosomes (PAC's)) and the like. Other vectors of interest include viral origin vectors (M13 vectors, bacterial phage λ vectors, baculovirus vectors, adenovirus vectors, and retrovirus vectors), high, low and adjustable copy number vectors, vectors which have compatible replicons for use in combination in a single host (e.g., pACYC184 and pBR322) and eukaryotic episomal replication vectors (e.g., pCDM8). The vectors contemplated by the invention include vectors containing inserted or additional nucleic acid fragments or sequences (e.g., recombinant vectors) as well as derivatives or variants of any of the vectors described herein.

Expression vectors useful in accordance with the present invention include chromosomal, episomal and virus derived vectors, e.g., vectors derived from bacterial plasmids or bacteriophages, and vectors derived from combinations thereof, such as cosmids and phagemids, and will preferably include at least one selectable marker (such as a tetracycline or ampicillin resistance genes) and one or more promoters such as the phage lambda P_{L} promoter, and/or the E. coli lac, trp and tac promoters, the T7 promoter and the baculovirus polyhedron promoter. Other suitable promoters will be known to the skilled artisan.

Among vectors preferred for use in the present invention include pQE70, pQE60 and pQE-9, available from Qiagen; pBS vectors, Phagescript vectors, Bluescript vectors, pNH8A, pNH16a, pNH18A, pNH46A, available from Stratagene; pcDNA3 available from Invitrogen; pGEX, pTrxfus, pTrc99a, pET-5, pET-9, pKK223-3, pKK233-3, pDR540, pRIT5 available from Pharmacia; and pSPORT1, pSPORT2 and pSV-SPORT1, available from Invitrogen Corporation. Other suitable vectors will be readily apparent to the skilled artisan.

Other terms used in the fields of recombinant DNA technology and molecular and cell biology as used herein will be generally understood by one of ordinary skill in the applicable arts.

The present invention provides novel microorganisms for biotechnology applications characterized by a more rapid growth rate than those microorganisms currently in use in the art. Both gram negative and gram positive prokaryotic cells may be used. The microorganisms of the present invention may be of any genus of microorganism known to those skilled in the art. The preferred characteristics of the microorganism are a rapid growth rate and the capability to be transformed with and to maintain exogenously applied DNA, in particular, to be transformed with and to maintain recombinant vectors. Rapid growing microorganisms of the present invention include, but are not limited to, microorganisms such as those of the genera Escherichia sp. (particularly E. coli), Klebsiella sp., Streptomyces sp., Streptocococcus sp., Shigella sp., Staphylococcus sp., Erwinia sp., Klebsiella sp., Bacillus sp. (particularly B. cereus, B. subtilis, and B. megaterium), Serratia sp., Pseudomonas sp. (particularly P. aeruginosa and P. syringae) and Salmonella sp. (particularly S. typhi or S. typhimurium). In a preferred embodiment, the microorganisms of the present invention are of the genus Escherichia. In other preferred embodiments, the microorganisms of the present invention may be of the species E. coli. In a preferred embodiment, the microorganisms of the present invention are E. coli strain W and derivatives thereof. The invention includes derivatives of E. coli strain W, including, e.g., E. coli strain W lacking endogenous plasmids and/or E. coli strain W lacking bacteriophage genetic material. In other preferred embodiments, the microorganisms of the present invention may be E. coli strains K, B or C, and derivatives thereof.

The microorganisms of the present invention may be identified by comparison to reference microorganisms. A preferred reference microorganism is E. coli MM294 (ATCC33625). Other suitable reference microorganism includes E. coli K-12 derived strains commonly used in molecular biology applications. The invention also contemplates any microorganism which grows at the same rate or at a faster rate when compared to the E. coli W strains of the present invention. Such comparison can be made by any means known to those skilled in the art, including time to colony formation and/or doubling time.

The microorganisms of the present invention preferably form colonies more rapidly than a reference microorganism. In particular, the microorganisms of the present invention will more rapidly form antibiotic resistant colonies after transformation with a vector containing an antibiotic resistance gene than the microorganisms of the prior art. To identify the microorganisms of the present invention, a putative rapid growing microorganism and a reference microorganism can, for example, be spread on suitable solid plates, preferably agar media plates known to those skilled in the art, in parallel. The selection and preparation of a suitable solid plate are within the capabilities of those skilled in the art. A suitable plate may prepared using the medium recommended by the American Type Culture Collection or other suitable media for cultivation of the candidate microorganism. Alternatively, a comparison of the doubling time in liquid media may be used.

The plates may optionally contain an antibiotic if, for example, a competent reference microorganism is to be compared to a competent, putative rapid growing microorganism. Both microorganisms can be transformed with a vector that confers an antibiotic resistance to transformants. After transformation, the two microorganisms can be spread onto antibiotic plates in parallel and incubated at an appropriate temperature. The time to the appearance of antibiotic resistant colonies of a specified diameter can be determined. The rapid growing microorganisms of the present invention will form antibiotic resistant colonies of a specified size more rapidly than the reference microorganism. The plates are incubated at the same temperature and the time to colonies of a specified size is determined. In the examples below, a colony size of 1 mm diameter was used; however, any size may be selected and used. A microorganism that attains the specified size at a faster rate than a reference organism is considered to be a rapid growing organism.

The invention includes rapid growing microorganisms that are free of bacteriophage infection. A rapid growing microorganism that is free of bacteriophage infection includes, e.g., a rapid growing microorganism that does not contain any bacteriophage genetic material or does not contain the genetic material of one or more particular bacteriophage types. In a preferred embodiment, the rapid growing bacterium is an E. Coli W that does not contain the genetic material of bacteriophage Wphi and/or does not contain the genetic material of bacteriophage Mu. Thus, the invention includes E. coli W that have been cured of both bacteriophage Wphi and bacteriophage Mu.

A bacterium is deemed to be free of bacteriophage infection or to lack the genetic material of one or more bacteriophage types when bacteriophage genetic material cannot be detected using standard nucleic acid detection methods known in the art. Exemplary nucleic acid detection methods for determining whether a bacterium is free of bacteriophage infection include, e.g., the polymerase chain reaction (PCR), Southern blotting and other nucleic acid hybridization methods involving the use of nucleic acid probes and/or primers. The nucleic acid detection method that is used will preferably be specific for a particular bacteriophage type. For example, a bacterium is deemed to lack the genetic material of bacteriophage Wphi when a nucleic acid detection method using probes or primers that are specific for a nucleic acid sequence found within the genetic material of Wphi does not produce a positive signal.

An alternative or additional method for determining whether a bacterium is free of bacteriophage infection is by plaque assay. For example, the putative bacteriophage-free bacterial strain can first be grown in liquid medium. The medium can then be removed and applied to a suitable test strain on solid medium (e.g., in agar overlay). Finally, the appearance of plaques can be monitored. The absence of plaques indicates that the putative strain is free of bacteriophage infection. Variations on the above-described plaque assay would be appreciated by those of ordinary skill in the art.

The expression “bacteriophage genetic material,” is intended to mean the nucleic acid-containing material derived from one or more bacteriophage types. Bacteriophage genetic material includes, e.g., bacteriophage-derived nucleic acid that is involved in, or that encodes elements involved in, one or more of the following processes: replication of bacteriophage nucleic acid, integration of bacteriophage nucleic acid into a bacterial chromosome, excision of bacteriophage nucleic acid from a bacterial chromosome, packaging of nucleic acid into virus particles, and lysis of bacterial cells.

A “bacteriophage type,” as used herein, is intended to include one or more bacteriophages that are distinguishable from other bacteriophages based on their genetic material and/or their virion morphology. Encompassed within the expression “bacteriophage type” are, e.g., bacteriophage with double stranded DNA genomes including, e.g., bacteriophage of the corticoviridae, lipothrixviridae, plasmaviridae, myrovridae, siphoviridae, sulfolobus shibate, podoviridae, tectiviridae and fuselloviridae families; bacteriophage with single stranded DNA genomes including, e.g., bacteriophage of the microviridae and inoviridae families; and bacteriophage with RNA genomes including, e.g., bacteriophage of the leviviridae and cystoviridae families. Exemplary bacteriophage types include, e.g., bacteriophage Wphi, Mu, T1, T2, T3, T4, T5, T6, T7, P1, P2, P4, P22, fd, phi6, phi29, phiC31, phi80, phiX174, SP01, M13, MS2, PM2, SSV-1, L5, PRD1, Qbeta, lambda, UC-1, HK97 and HK022. The expressions “bacteriophage” and “phage” may be used interchangeably.

In the context of the present invention, bacteria that do not contain the genetic material of one or more bacteriophage type, and bacteria that do not contain any bacteriophage genetic material, are those bacteria that do not contain bacteriophage genetic material either integrated into the bacterial chromosome or otherwise present (e.g., in packaged or unpackaged form) within the confines of the bacterial cell membrane and/or cell wall.

In certain embodiments of the invention, rapid growing bacteria are provided that lack endogenous plasmids and also do not contain the genetic material of one or more specified bacteriophage types or do not contain any bacteriophage genetic material. For example, the invention includes E. coli W that lack endogenous plasmids and that do not contain the genetic material of bacteriophage Wphi and/or do not contain the genetic material of bacteriophage Mu.

The invention also provides methods for producing rapid growing bacteria that do not contain any bacteriophage genetic material or that do not contain the genetic material of one or more specified bacteriophage types. For example, the invention provides methods for curing rapid growing bacteria of bacteriophage nucleic acid. Bacteriophage nucleic acid may integrate into the chromosome of a bacterial host cell. In certain cases, the location on the bacterial chromosome at which a particular bacteriophage integrates is known. Alternatively, the site of integration may be random. Whether a bacteriophage inserts its nucleic acid at a specific point on a bacterial chromosome or at random locations will influence the methods that are used by one of skill in the art to produce bacteriophage-free bacteria. Methods for curing a bacterium of bacteriophage genetic material are well known in the art. For example, bacteria can be cured of bacteriophage nucleic acid using transposon-mediated methods. See, e.g., Kleckner, N. et al., Methods Enzymol. 204:139-180 (1991). Tn10, for instance, is a transposon that can be used to produce rapid growing bacteria that do not contain the genetic material from one or more bacteriophage type. See id. Alternatively, recombinational cloning methods can be used to produce bacteria that lack bacteriophage genetic material. (Zhang, Y., et al., Nat. Genet. 20:123-128 (1998), Datsenko, K. A. and Wanner, B. L., Proc. Natl. Acad. Sci. USA 97:6640-6645 (2000), Skorupski, K. and Taylor, R. K., Gene 169:47-52 (1996).

Integrated bacteriophage genetic material can be deleted from a bacterial genome by, e.g., homologous recombination using a nucleic acid molecule carrying a selectable marker flanked by sequences of about 30 to about 60 nucleotides that are homologous to the region flanking the integration site of the bacteriophage genetic material in the host genome. See WO 99/29837. The nucleic acid molecule used to delete the bacteriophage genetic material may alternatively be comprised of, e.g., two homology arms, each about 30 nucleotides in length, flanking a single FRT site. See WO 2002/014495.

The nucleic acid molecules used in these methods may be generated using, e.g., the polymerase chain reaction (PCR). The nucleic acid molecules can be introduced into the bacterial cell by, e.g., electroporation or other transformation techniques, and the recombination step can be mediated using lambda recombination functions (αβγ). Excision can be accomplished by expression of FLP recombinase.

According to another exemplary method, integrated bacteriophage genetic material can be deleted from a bacterial genome through the use of recA-mediated recombination. For example, bacteriophage genetic material can be deleted via the use of recA-mediated recombination and a “suicide” plasmid. The suicide plasmid contains a gene which confers sensitivity to a particular substance. For example, the suicide plasmid may contain the rpsL gene which confers sensitivity to streptomycin. The suicide plasmid also contains two sequences, about 600 nucleotides in length each, that are homologous to the ends of the bacteriophage genetic material and to the sites of bacteriophage integration. These two sequences flank a FRT-[drug-resistance gene]-FRT cassette in the suicide plasmid. The drug resistance gene can be any known gene which confers resistance to substances that ordinarily kill or impair the growth of bacteria such as the suicide plasmid is introduced into the bacterial host cell and will insert at the site of bacteriophage integration. The FRT-[drug-resistance gene]-FRT cassette is excised with FLP recombinase leaving a single FRT site in place of the bacteriophage genetic material.

Other methods known in the art for removing specific sequences from a bacterial genome can be used in conjunction with the present invention in order to cure rapid growing bacteria of bacteriophage infection.

In one embodiment, the invention includes methods for curing E. coli W of bacteriophage Wphi genetic material. In another exemplary embodiment, the invention includes methods for curing E. coli W of bacteriophage Mu genetic material.

The present invention also includes rapid growing bacteria that are resistant to infection by one or more specified bacteriophage types. Also included are methods for producing bacteriophage-resistant rapid growing bacteria. Bacteria that are resistant to infection by bacteriophage, within the context of the present invention, include bacteria having cellular properties that inhibit or substantially reduce the ability of one or more bacteriophage types to insert their genetic material into the bacterial cell. Bacteriophage-resistant rapid growing bacteria include, e.g., bacteria whose cell surface possesses properties such that one or more type of bacteriophage cannot attach to the cell surface and/or are not able to insert bacteriophage nucleic acid into the cytoplasm of the bacterial cell.

Rapid growing bacteria of the invention can be made resistant to bacteriophage infection by, e.g., introducing certain mutations into the bacterial genome. Mutations that render bacterial strains resistant to bacteriophage infection are known in the art and can be introduced into the rapid growing bacteria of the present invention using well-known methods. Exemplary mutations that cause resistance to bacteriophage include mutations in tonA, tonB and in the gene encoding the lambda receptor. Bacteriophage resistance can also be obtained by genetic screening. Bacteriophage resistance is further described, e.g., in U.S. Pat. Nos. 5,538,864, 5,432,066 and 5,658,770 and in Saito, H. and Richardson, C. C., J. Virol. 37:343-352 (1981), Stacey, K. A. and Oliver, P., J. Gen. Microbiol. 98:569-578 (1977), Coulton, J. W., Biochim. Biophys. Acta 717:154-162 (1982), Carta, G. R. and Bryson, V., J. Bacteriol. 92:1055-1061 (1966), Ronen A. and Zehavi, A., J. Bacteriol. 99:784-790 (1969), Braun-Breton, C. and Hofnung, M., Mol. Gen. Genet. 16:143-149 (1978), and Picken, R. N. and Beacham, I. R., J. Gen. Microbiol. 102:305-318 (1977).

The present invention also comprises a method of cloning employing the rapid growing microorganisms of the present invention. A vector or a population of recombinant vectors containing a desired insert may be constructed using techniques known in the art. For example, DNA of interest may be digested with one or more restriction enzymes to generate a fragment. The fragment may be purified on an agarose gel. A vector is prepared by digestion with the appropriate restriction enzymes. The vector may be further treated with other enzymes such as alkaline phosphatase or the Klenow fragment of DNA polymerase, and may be gel purified. The DNA fragment is ligated into the vector using an appropriate ligase enzyme to generate a population of recombinant vectors. The DNA of interest may be a cDNA obtained by, e.g., RT-PCR.

Other methods to produce a vector or a population of recombinant vectors may be used. For example, a population of recombinant vectors may be produced by recombinational cloning. An insert donor molecule is prepared comprising a DNA of interest flanked by a first and a second recombination site, wherein the first and the second recombination site do not recombine with each other. The insert donor molecule is contacted with a vector donor molecule comprising a third and a fourth recombination site, wherein the third and the fourth recombination sites do not recombine with each other. The insert donor/vector donor mixture is further contacted with one or more site specific recombination proteins capable of catalyzing recombination between the first and the third recombination sites and/or the second and the fourth recombination sites thereby allowing recombination to occur and generating a population of recombinant vectors.

Once constructed, the vector or the population of recombinant vectors is introduced into competent, rapid growing microorganisms using any one of the many techniques for the introduction of vector into a microorganism known to those skilled in the art. The transformed microorganisms are grown and recombinant microorganisms, i.e. those containing a vector, are selected. In one embodiment, the genotype of the microorganism is suitable for screening by alpha complementation and the selection step may include the use of a blue/white screen on solid plates containing a chromogenic substrate for β-galactosidase, such as X-gal. The vectors are isolated from the recombinant microorganism and analyzed for the presence of the DNA of interest.

The present invention also comprises a method of producing a desired protein or peptide utilizing the rapid growing microorganisms of the present invention. The method comprises constructing a recombinant vector containing a gene encoding the desired protein, transforming the vector into a competent, rapid growing microorganism and culturing the transformed microorganism under conditions that cause the transformed microorganism to produce the desired protein. The recombinant vector may be constructed using the methodology described above. In one embodiment, the recombinant vector will include an inducible promoter to control transcription from the gene coding for the desired protein. In other preferred embodiments, the genome of the microorganism will contain a gene for the T7 RNA polymerase under the control of an inducible promoter. In other preferred embodiments, the promoter controlling the expression of the T7 RNA polymerase will be inducible by the addition of salt to the growth media. In other preferred embodiments, the promoter controlling the expression of the T7 RNA polymerase will be regulated by the addition of arabinose to the growth media.

In preferred embodiments, the rapid growing microorganism is of the genus Escherichia. In other preferred embodiments, the rapid growing microorganism is an E. coli. In other preferred embodiments, the rapid growing microorganism is an E. coli strain W. In another preferred embodiment, the rapid growing microorganism does not contain endogenous plasmids. In another preferred embodiment, the rapid growing microorganism is a bacterium that does not contain any bacteriophage genetic material or that does not contain the genetic material of one or more specified bacteriophage types. In another preferred embodiment, the rapid growing microorganism is a bacterium that is resistant to infection by one or more bacteriophage type.

In other preferred embodiments, the genotype of the microorganism has been altered to inactivate one or more genes coding for a protease and/or a ribonuclease. In one such preferred embodiment, the rapid growing microorganism does not contain a functional ion protease and/or a functional ompT protease. In other preferred embodiments, the rapid growing microorganism of the present invention does not have a functional rnaE gene and/or a functional rnaI gene. In other preferred embodiments the microorganism does not contain functional ion protease and/or a functional ompT protease and does not contain a functional ribonuclease encoded by the rnaE gene and/or the rnaI gene.

The present invention also includes rapid growing microorganisms that are unable to synthesize components of the cell membrane and therefore are unable to grow in media lacking the particular cell membrane component. For example, the invention includes rapid growing bacteria, e.g., rapid growing E. coli that are unable to synthesize diaminopimelic acid and therefore are unable to grow in media that lacks diaminopimelic acid. Such rapid growing bacteria are useful, e.g., in methods that involve the negative selection of cells that do not contain a desired exogenous plasmid. For example, rapid growing E. coli that are unable to synthesize diaminopimelic acid, e.g., a dap⁻ strain, can be transformed with a plasmid of interest that comprises, along with other desired elements, a gene that renders the strain capable of growing in the absence of diaminopimelic acid. For instance, a wild-type Dap gene provided on a plasmid of interest will allow a dap⁻ strain to grow in media lacking diaminopimelic acid. By culturing the E. coli cells that have been transformed with the plasmid of interest in media lacking diaminopimelic acid, a strong negative selection will be imposed against cells that have not received the plasmid. This system is particularly useful when using rapid growing E. coli since other selection systems (e.g., systems involving the use of ampicillin) may be less effective due to the rapid growth characteristics of the rapid growing E. coli.

Accordingly, the present invention also includes methods for selecting for rapid growing bacteria that contain a plasmid of interest, said method comprising: (a) obtaining rapid growing bacteria that are unable to grow in media lacking diaminopimelic acid, (b) transforming said rapid growing bacteria with a plasmid comprising a gene that restores the ability of said rapid growing bacteria to grow in the absence of diaminopimelic acid, and (c) culturing the transformed rapid growing bacteria in medium lacking diaminopimelic acid. In an exemplary embodiment, the rapid growing bacteria are E. coli having a growth rate that is at least 5%, 25%, 50%, 100%, or 200% greater than the growth rate of E. coli MM294. In a preferred embodiment, the rapid growing bacteria are E. Coli strain W. The bacteria useful in this aspect of the invention may, in certain embodiments, be free of endogenous plasmids and/or may not contain any bacteriophage genetic material or may not contain the genetic material of one or more specified bacteriophage types. The bacteria useful in this aspect of the invention may also be resistant to infection by one or more bacteriophage type.

The present invention also includes kits comprising a carrier or receptacle being compartmentalized to receive and hold therein at least one container, wherein the container contains rapid growing microorganisms. The kit optionally further comprises vectors suitable for cloning. In a preferred embodiment, the kits may contain a vector suitable for recombinational cloning. Optionally, the kits of the present invention may contain enzymes useful for cloning. In a preferred embodiment, the kits may contain one or more recombination proteins. In a preferred embodiment, the rapid growing microorganisms may be competent. In some preferred embodiments, the rapid growing microorganisms may be chemically competent. In other preferred embodiments, the rapid growing microorganisms may be electrocompetent.

The present invention includes compositions comprising rapid growing microorganisms. In a preferred embodiment, the rapid growing microorganism may be a competent microorganism. In some preferred embodiments, the rapid growing microorganisms may be chemically competent. In other preferred embodiments, the rapid growing microorganisms may be electrocompetent. The compositions of the present invention may optionally comprise at least one component selected from buffers or buffering salts, one or more DNA fragments, one or more vectors, one or more recombinant vectors, one or more recombination proteins and one or more ligases. In a preferred embodiment, the compositions of the present invention may comprise a rapid growing microorganism in a glycerol solution. In other preferred embodiments, compositions of the present invention may comprise rapid growing microorganisms in a buffer. In preferred embodiments, the microorganisms of the present invention may be in a competence buffer. In other preferred embodiments, the compositions of the present invention may comprise a lyophilized rapid growing microorganism.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLE 1

### Strain Construction

All strains (listed in table 1) were constructed via bacteriophage P1 mediated transduction (Jeffrey Miller, Experiments in Molecular Genetics, Cold Spring Harbor Laboratories, 1972, specifically incorporated herein by reference). E. coli strains containing Tn10 insertions suitable for use with the P1 transduction technique can be obtained from the University of Wisconsin. The parental strain for this work was an E. coli W strain designated ATCC9637 obtained from the American Type Culture Collection (Manassas, Va.). The isolate received was resistant to bacteriophage P1. ATCC9637 was, therefore, converted to a P1 sensitive phenotype by infection with bacteriophage P1Cmts. P1Cmts is a bacteriophage P1 derivative which contains a temperature sensitive repressor and contains the chloramphenicol resistance gene. The bacteriophage forms P1 lysogens at 30° C. but replicates lytically at higher temperatures (>37° C.). E. coli W ATCC9637 was mixed with bacteriophage P1Cmts and chloramphenicol resistant colonies (which are P1Cm lysogens) were selected on LB chloramphenicol plates at 30° C. The chloramphenicol resistant strain was cured of the P1 lysogen by selection for surviving colonies at 42° C. The surviving colonies are now chloramphenicol sensitive. The P1 sensitive derivative of ATCC9637 (BRL3234) was then used for all subsequent work.

TABLE 1
LIST OF STRAINS USED IN THE EXPERIMENTS

| | | Plasmids | | | |
|---|---|---|---|---|---|
| Strain | Relevant Genetic Markers | 5.5 kb | 6.5 kb | >50 kb | Other |
| E. coli W | | | | | |
| ATCC9637 | | + | | + | |
| BRL3234 | P1 sensitive of ATCC9637 | | | | |
| BRL3234/pCM301 | amp resistant temp sensitive | | | | pCM301 |
| BRL3573 | nupG::Tn10 endA⁻ | | | | pCM301 |
| BRL3574 | nupG::Tn10 endA⁻ | | | | |
| BRL3580 | endA⁻ | | + | + | |
| BRL3582 | As 3580 endA srl:: Tn10 recA | | | | |
| | deletion 1398 | | | | |
| BRL3711 | endA⁻ | | +(Apr) | + | |
| BRL3718 | endA⁻ | | − | + | |
| BRL373410 | endA⁻ | | | +(Cm^{r}) | |
| BRL3741 | As 3718 | | − | − | deletion |
| | | | | | 1 (Km^{r}) |
| BRL3742 | As 3718 | | − | − | deletion |
| | | | | | 3 (Km^{r}) |
| BRL3756 | SDS curing of 3741 | | − | − | |
| BRL3757 | SDS curing of 3742 | | − | − | |
| BRL3745 | As 3734 | | − | − | deletion |
| | | | | | 1 (Km^{r}) |
| BRL3746 | As 3734 | | − | − | deletion |
| | | | | | 3 (Km^{r}) |
| BRL3762 | SDS curing of 3745 | | − | − | |
| BRL3763 | SDS curing of 3746 | | − | − | |
| BRL3759 | DH10B recA⁺ zah281::Tn10 | | | | |
| | lacX74 | | | | |
| BRL3764 | DH10B recA⁺ trp-::Tn10 | | | | |
| | φ80dlacZΔM15 | | | | |
| BRL3760 | lacX74 zah281::Tn10 | | | | |
| BRL3761 | lacX74 zah281::Tn10 | | | | |
| BRL3766 | lacX74 | | | | |
| BRL3766/pSU39 | lacX74 | | | | pSU39 |
| BRL3769 | lacX74 | | | | |
| BRL3769/pSU39 | lacX74 | | | | pSU39 |
| BRL3776 | lac74 trp-::Tn10 | | | | pSU39 |
| | φ80dlacZΔM15 | | | | |
| BRL3778 | lac74 trp-::Tn10 | | | | pSU39 |
| | φ80dlacZΔM15 | | | | |
| BRL3781 | lac74 trp-::Tn10 | | | | |
| | φ80dlacZΔM15 | | | | |
| BRL3776 | lac74 trp-::Tn10 | | | | |
| | φ80dlacZΔM15 | | | | |
| E. coli K-12 | | | | | |
| DB3.2 | nupG::Tn10 endA⁻ | | | | |
| DH10B | endA⁻ | | | | |
| BRL3709 | As DH10B | | +(Ap^{r}) | | |
| BRL3726 | As DH10B | | | +(Km^{r}) | |
| BRL3727 | As DH10B | | | +(Cm^{r}) | |
| DH5α | | | | | |
| BRL3740(1) | As DH5α | | | | deletion |
| | | | | | 1 (Km^{r}) |
| BRL3740(3) | As DH5α | | | | deletion |
| | | | | | 3 (Km^{r}) |
| E. coli C | | | | | |
| BRL3229 | E. coli C srl-::Tn10 recA | | | | |
| | deletion | | | | |

### EXAMPLE 2

### Construction of E. coli W endA⁻

Competent cells of BRL3234 were prepared by a modification of the method of Hanahan (Doug Hanahan, J. Mol. Biol. 166,557, 1983) as described in U.S. Pat. No. 4,981,797 which is specifically incorporated herein by reference. The competent cells were transformed with pCM301 plasmid DNA (Tucker, et al., 1984, Cell 38(1):191-201.), a plasmid which is temperature sensitive for replication. Transformants were selected on ampicillin plates at 30° C. The introduction of the pCM301 plasmid into BRL3234 aided in the identification of endA⁻ derivatives as described below.

Bacteriophage Plvir was grown on an E. coli strain, DB2, which contains an endA mutation linked to the nupG::Tn10 transposon. The P1 lysate grown on DB3.2 was used to infect BRL3234/pCM301 with selection for tetracycline resistance. The tet^{r} colonies were then screened for the linked endA⁻ mutation by determining the ability of the transductants to degrade the pCM301 DNA after preparation of miniprep DNA. Those transductants which degraded the plasmid DNA were endA⁺ and those which did not degrade pCM301 plasmid DNA were endA⁻. The tetracycline resistant, endA⁻ derivative of BRL3234/pCM301 was designated BRL3573. A derivative of BRL3573 lacking pCM301 was selected by streaking BRL3573 on an LB plate at 42° C. and screening colonies for ampicillin sensitivity.

The ampicillin sensitive derivative of BRL3573 was designated BRL3574. The nupG::Tn10 transposon was cured from BRL3574 using LB plates containing fusaric acid (Stanley Maloy and William Nunn, J. Bacteriol. 145:1110, 1981). One tetracycline sensitive derivative of BRL3574 was designated BRL3580. BRL3580 is E. coli W endA⁻.

### EXAMPLE 3

Construction of BRL3582 a recA⁻ E. coli W.

A P1Cm lysate was grown on BRL3229. BRL3229 contains a Tn10 transposon linked to a deletion mutation in recA. The P1 lysate was used to transduce BRL3580 and tetracycline resistant transductants were selected at 30° C. on LB plates containing 20 μg/mL tetracycline. The transductants were re-purified once on LB tetracycline plates and were then screened for sensitivity or resistance to nitrofurantoin on LB plates containing 4 μg/mL nitrofurantoin. RecA⁺ strains are resistant to nitrofurantoin whereas recA⁻ strains are sensitive to nitrofurantoin (S Jenkins and P. Bennett J., Bacteriol. 125:1214, 1976). One tetracycline resistant, nitrofurantoin sensitive derivative of BRL3580 was designated BRL3582.

### EXAMPLE 4

### Isolation of E. coli W Derivatives Lacking Native Plasmids

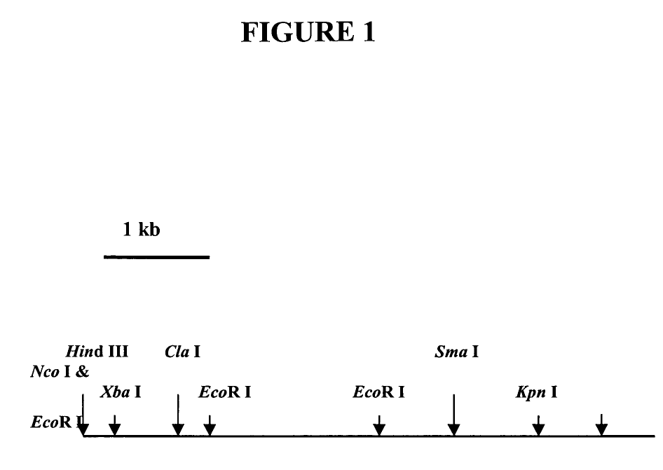

ATCC9637 and all strains derived from ATCC9637 up to and including BRL3580 contain 2 plasmids. The smaller plasmid is approximately 5.5 kb and the larger plasmid is >50 kb. The 5.5 kb plasmid was prepared from ATCC9637 by Lofstrand Labs (Gaithersburg, Md.). A restriction map of this plasmid is provided in FIG. 1.

The restriction map provided cloning sites which could be used to introduce a gene conferring resistance to ampicillin. The ampicillin resistance gene was isolated from plasmid pTrcN2, a pProEX-1 derivative Invitrogen Corporation). The source of the ampicillin resistance gene is not critical. The following protocol will work with pProEX-1 and may be modified by those skilled in the art depending on the plasmid used as a source of the ampicillin resistance gene. 1 μg of plasmid pTrcN2 was digested with BspH1 (New England Biolabs) and the ends filled in with Klenow (Invitrogen Corporation). The 1008 bp DNA fragment containing the ampicillin resistance gene was purified by agarose gel electrophoresis. The 5.5 kb plasmid was digested with SmaI (New England Biolabs) and then treated with TsAP, a temperature sensitive alkaline phosphatase (Invitrogen Corporation). The DNAs were mixed, treated with T₄ DNA ligase (Invitrogen Corporation) and transformed into competent ME DH10B cells (Invitrogen Corporation). Ampicillin resistant colonies were selected on LB plates containing 100 μg/mL ampicillin. Several ampicillin resistant colonies were grown in overnight culture and plasmid DNA was prepared and analyzed by electrophoresis on an agarose gel. All ampicillin resistant clones were found to have a plasmid with a molecular weight of 6.5 kb. The DH10B cells containing the plasmid (designated Wamp) were designated BRL3709.

The Wamp plasmid was transformed into competent cells of BRL3580 (E. coli W endA⁻) with selection for ampicillin resistance. BRL3580, as well as 5 ampicillin resistant transformants, were grown at 37° C. in LB broth containing 100 μg/mL ampicillin and the plasmid DNA was isolated and analyzed by agarose gel electrophoresis. The plasmid DNA from BRL3580 had a molecular weight of 5.5 kb whereas the ampicillin resistant transformants had plasmid DNA with a molecular weight of 6.5 kb indicating that the ampicillin resistance gene ˜1 kb had been introduced into the 5.5 kb plasmid to give a 6.5 kb plasmid. Further, the 6.5 kb plasmid containing the ampicillin resistance gene had displaced the 5.5 kb plasmid. This is the expected result since both plasmids contained the same origin of replication. The E. coli W derivatives containing the 6.5 kb Wamp plasmid were designated BRL3711. Both BRL3580 and BRL3711 also contained the higher molecular weight (>50 kb) plasmid.

### EXAMPLE 5

Curing BRL3711 of the 6.5 kb Wamp Plasmid

BRL3711 was cured of the Wamp plasmid by growth in LB broth containing SDS. SDS is well known in the literature as a compound which is used to cure plasmids from E. coli strains (A. Bharathi and H. Polasa, FEMS Microbiol. Lett, 84:37, 1991, Susana Rosos, Aldo Calzolari, Jose La Torre, Nora Ghittoni, and Cesar Vasquez, J. Bacteriol 155:402, 1983). BRL3711 was grown in LB broth containing 10% SDS at 30° C. After the culture reached the stationary phase, the culture was diluted 1:1000 into fresh LB+10% SDS for a second cycle. After the second cycle, the survivors were plated on LB plates 30° C. and colonies were screened for sensitivity to ampicillin. One isolate, designated BRL3718, was found to be sensitive to ampicillin indicating that the 6.5 kb plasmid had been cured. Miniprep DNA derived from BRL3711 as well as BRL3718 confirmed that BRL3711 had both the smaller and larger plasmids but that BRL3718 had only the larger plasmid.

### EXAMPLE 6

### Preparation of a Derivative of the Large Plasmid Containing an Antibiotic Resistance Gene.

To isolate E. coli W derivatives lacking the larger plasmid, antibiotic resistance genes were introduced into the larger plasmid using the Genome Primer System from New England BioLabs. The larger plasmid was isolated from BRL3718 using the standard alkaline-SDS lysis procedure (J. Sambrook, E.F. Fritsch, and T. Maniatis. 1989 Molecular Cloning: A Laboratory Manual 2^{nd} Ed. Cold Spring Harbor Laboratory Press. Cold Spring Harbor N.Y.). The Genome Priming System was used according to instructions provided by the manufacturer.

Approximately 80 ng of target plasmid DNA was mixed with 20 ng of donor plasmid DNA in a 20 μL reaction. One donor plasmid, pGSP1, donates the gene conferring resistance to kanamycin (Km). The second donor plasmid, pGSP2, donates the gene conferring resistance to chloramphenicol (Cm). The final reactions were diluted 1:10 in water and electroporated into EMax DH10B cells (Invitrogen Corporation). 20 μL of cells were mixed with 1 μL of the diluted reaction and the cell-DNA combination was electroporated at 420 V, 4000 ohms, 2.4 kV, 16000 kV/cm. 10 μL were expressed in 1 mL SOC for 1 hour 37° C. 100 μL of the expression mix were plated on LB plates containing either 10 μg/mL kanamycin for the pGPS1 reaction or LB plates containing 12.5 μg/mL chloramphenicol for the pGPS2 reaction. 8 transformants from each reaction were analyzed. Plasmid DNA from all 16 colonies had a high molecular weight plasmid which ran on an agarose gel in approximately the same position as the plasmid DNA isolated from BRL3718. In addition, several of the plasmid DNAs were again electroporated into EMax DH10B cells and were shown to confer resistance to either kanamycin or chloramphenicol on the DH10B cells. It was concluded that the genes conferring resistance to either kanamycin or chloramphenicol had been introduced into the large molecular weight plasmid from BRL3718. DH10B cells containing the high molecular weight plasmid which confers resistance to kanamycin have been designated BRL3726. DH10B cells containing the high molecular weight plasmid which confers resistance to chloramphenicol have been designated BRL3727.

### EXAMPLE 7

### Construction of Deletion Plasmids.

Plasmid DNA from the strain BRL3726 (DH10B containing the high MW plasmid+KM^{r} marker) was prepared. In two separate reactions, 1 μg of plasmid DNA was partially digested with 0.5 and 0.1 units of the restriction enzyme Sau3A I (Invitrogen Corporation) at 37° C. for 15 min. The reactions were extracted with phenol/chloroform and precipitated with ethanol. The DNA from each reaction was ligated using T₄ DNA Ligase (Invitrogen Corporation) and transformed into competent ME DH5α cells (Invitrogen Corporation). Colonies were selected on LB plates containing 20 μg/mL kanamycin at 37° C. Chemically competent cells were used because they are not as efficient in taking up high molecular weight plasmid DNA as electrocompetent cells.

The plasmid DNA from 10 kanamycin resistant (from the 0.1 U reaction) colonies was analyzed by agarose gel electrophoresis. The size of the deletion plasmid DNA ranged from ˜4.5-15 kb and the plasmids were designated deletion 1-deletion 10. DH5α cells containing these plasmids were designated BRL3740-1 to BRL3740-10.

### EXAMPLE 8

### Curing BRL3718 of the High Molecular Weight Plasmid DNA.

Chemically competent cells of BRL3718 were prepared according to the method of Hanahan (Hanahan D., 1983 J. Mol Biol 166,557) as modified according to U.S. Pat. No. 4,981,797. Chemically competent cells of BRL3718 were transformed with plasmid DNA isolated from BRL3740-1 (deletion 1, ˜8 kb) and BRL3740-3 (deletion 3, ˜10 kb) and kanamycin resistant colonies were selected on LB plates containing 20 μg/mL kanamycin at 37° C. Four colonies from each transformation were streaked for single-colony isolates onto LB plates containing 20 μg/mL kanamycin at 37° C. Plasmid DNA was isolated from 4, single-colony isolates and analyzed by agarose gel electrophoresis.

The high molecular weight plasmid DNA was readily apparent in miniprep DNA prepared from BRL3718. However, plasmid DNA prepared from the kanamycin resistant transformants did not indicate the presence of the high molecular weight plasmid DNA. Rather, plasmid DNAs with molecular weights characteristic of BRL3740-1 (˜8 kb) and BRL3740-3 (˜10 kb) were readily visible. It was concluded that the transformation of deletion 1 and deletion 3 plasmid DNA into BRL3718 resulted in replacement of the high molecular weight plasmid DNA (>50 kb) with deletion 1 and deletion 3 DNA. This is the expected result since the high molecular weight plasmid DNA, deletion 1 plasmid DNA and deletion 3 plasmid DNA all share the same origin of replication. The BRL3718 derivatives containing deletion 1 and deletion 3 plasmid DNA were designated BRL3741 and BRL3742, respectively.

### EXAMPLE 9

### Curing BRL3741 and 3742 of the Kmr Plasmids.

BRL3741 and BRL3742 were grown overnight in LB broth containing 10% SDS at 30° C. The cultures were diluted 1:1000 into LB broth containing 10% SDS and incubated again at 30° C. After 2 cycles at 30° C., dilutions of these cultures (1:104 and 1:106) were applied to LB plates, incubated at 30° C., and screened for sensitivity to kanamycin. For BRL3741, 15/50 colonies were sensitive to kanamycin while 9/50 colonies from BRL3742 were sensitive to kanamycin. Plasmid DNA from 2 kanamycin sensitive derivatives of BRL3741 and 2 kanamycin sensitive derivatives of BRL3742 was isolated and analyzed by agarose gel electrophoresis. No plasmid DNA corresponding to the deletion plasmids was observed on the gel. The BRL3741 derivatives cured of the deletion 1 plasmid were designated BRL3756. The BRL3742 derivatives cured of the deletion 3 plasmid were designated BRL3757.

### EXAMPLE 10

### Competent Cells of BRL3756 and BRL3757.

Chemically competent cells of BRL3741, BRL3742, BRL3756 and BRL3757 were prepared according to the method of Hanahan (Hanahan D., 1983 J. Mol Biol 166,557) as modified according to U.S. Pat. No. 4,981,797. BRL3741 and BRL3742 were streaked on LB plates containing 20 μg/mL kanamycin and the plates were incubated at 28° C. for 20 hours. BRL3756(1), BRL3756(2), BRL3757(1) and BRL3757(2) were streaked on LB plates and the plates were incubated 28° C. for 20 hours. 5-6 colonies of each strain were picked into 1 mL SOB medium (D, Hanahan J. Mol Biol 166:557 1983). 0.9 mL of the cells were inoculated into 60 mL SOB medium in a 500 mL baffled shake flask. The flasks were placed in an 28° C. incubator 250 rpm. When the OD at 550 nm reached 0.25-0.33, the cells were harvested. 50 mL of cells of each strain were centrifuged (4° C.) and the cells were re-suspended in 4 mL cold CCMB80 buffer (D. Hanahan, J. Jessee and F. Bloom Methods in Enzymology 204:63 1991, specifically incorporated herein by reference). The cells were allowed to sit on ice for 20 min. 220 μL were placed in NUNC vials and the cells were frozen in a dry ice ethanol bath. The cells were stored at −70° C.

### EXAMPLE 11

### Evaluation of Time to Ampicillin Resistant Colony.

Vials of competent cells (ATCC9637, BRL3718, BRL3741, BRL3742, BRL3756 and BRL3757) were thawed on ice for 20 min. 100 μL of cells were mixed in a cold Falcon 2059 tube with pUC19 (5 μL of 10 pg/μL=50 pg). The cells were allowed to sit on ice for 15 min. The cells were heat shocked at 42° C. for 45 seconds followed by a 2 minute incubation on ice. 0.9 mL of room temperature SOC was added to each tube and the tubes were shaken at 37° C. (250 rpm) for 30 minutes. Aliquots of the expression mix were plated on LB plates containing 100 μg/mL ampicillin and the plates were incubated at either 42° C. or 37° C. The time to the appearance of 1 mm colonies is shown in table 2. At 37° C., ampicillin resistant colonies of 1 mm size required between 7.8 and 8.2 hours and there was no significant difference in time between strains containing both the 5.5 kb plasmid and the >50 kb plasmid (ATCC9637), strains containing only the >50 kb plasmid (BRL3718), strains containing the smaller kanamycin resistant plasmid (BRL3741 and 3742), or strains containing no plasmids (BRL3756 and 3757). In fact at 42° C. colonies of 1 mm size required 7.7 hours for all strains tested. It was concluded that the presence or absence of plasmids in E. coli W does not significantly affect the time to appearance of colonies after transformation.

TABLE 2
TIME IN HOURS TO AMPICILLIN RESISTANT COLONIES
AFTER TRANSFORMATION WITH PUC19 DNA.

Time to 1 mm
colony size

| STRAIN | 42° C. | 37° C. |
|---|---|---|
| ATCC 9637 | 7.7 | 8.2 |
| BRL3718 | 7.7 | 7.8 |
| BRL3741 | 7.7 | 8.2 |
| BRL3742 | 7.7 | 7.8 |
| BRL3756 (1) | 7.7 | 8.2 |
| BRL3756 (2) | 7.7 | 8.2 |
| BRL3757 (1) | 7.7 | 7.8 |
| BRL3757 (2) | 7.7 | 8.2 |

### EXAMPLE 12

### Construction of BRL3734.

Electrocompetent cells of BRL3718 were prepared according to a modification of the protocol described in Hanahan et. al., Methods in Enzymology, vol. 204, p. 63 (1991). DNA from BRL3727 isolate 4₆ was used to introduce the plasmid into BRL3718. 20 μL of cells were mixed with 1 μL of DNA and the cell-DNA mixture was electroporated at 250 V, 2000 ohms, 1.44 kV, 9.6 kV/cm in the Cell-Porator. 10 μL were expressed in 1 mL SOC for 60 min 37° C. and the expression was plated on LB plates containing 12.5 μg/mL chloramphenicol. After 24 hours the colonies were re-purified and analyzed. The miniprep DNA contained a plasmid with a molecular weight approximately the same size as the plasmid found in BRL3718. The E. coli W strain containing the chloramphenicol resistant high molecular weight plasmid was designated BRL3734.

### EXAMPLE 13

### Curing BRL3734 of the High Molecular Weight Plasmid DNA.

Chemically competent cells of BRL3734 were prepared according to the method of Hanahan (Hanahan D., 1983 J. Mol Biol 166,557) as modified according to U.S. Pat. No. 4,981,797. Chemically competent cells of BRL3734 were transformed with plasmid DNA isolated from BRL3740-1 (deletion 1, ˜8 kb) and BRL3740-3 (deletion 3, ˜10 kb) and kanamycin resistant colonies were selected on LB plates containing 20 μg/mL kanamycin at 37° C. Four colonies from each transformation were streaked for single-colony isolates onto LB plates containing 20 μg/mL kanamycin at 37° C. Plasmid DNA was isolated from 4, single-colony isolates and analyzed by agarose gel electrophoresis. The high molecular weight plasmid DNA was readily apparent in miniprep DNA prepared from BRL3734. However, plasmid DNA prepared from the kanamycin resistant transformants did not indicate the presence of the high molecular weight plasmid DNA. Rather, plasmid DNA with molecular weight characteristic of BRL3740-1 (˜8 kb) and BRL3740-3 (˜10 kb) were readily visible. Moreover, BRL3734 containing deletion 1 and deletion 3 plasmids were streaked for single-colony isolates onto LB containing Km 20 μg/mL and LB containing Cm 12.5 μg/mL plates to confirm the presence, or absence, of the desired plasmid DNAs. No growth was observed on the LB+Cm 12.5 μg/mL plates while the formation of single-colony isolates was observed on Km 20 μg/mL plates. It was concluded that the transformation of deletion 1 and deletion 3 plasmid DNA into BRL3734 resulted in replacement of the high molecular weight plasmid DNA (>50 kb) with deletion 1 and deletion 3 DNA. This is the expected result since the high molecular weight plasmid DNA, deletion 1 plasmid DNA and deletion 3 plasmid DNA all share the same origin of replication. The BRL3734 derivatives containing deletion 1 and deletion 3 plasmid DNA were designated BRL3745 and BRL3746, respectively.

### EXAMPLE 14

### Curing BRL3745 and 3746 of the Kmr Plasmids.

BRL3745 and BRL3746 were grown overnight in LB broth containing 10% SDS at 30° C. The cultures were diluted 1:1000 into LB broth containing 10% SDS and incubated again at 30° C. After 2 cycles at 30° C., dilutions of these cultures (1:10⁶) were applied to LB plates, incubated at 30° C., and screened for sensitivity to kanamycin. For BRL3745, 22/100 colonies were sensitive to kanamycin while 1/100 colonies from BRL3742 were sensitive to kanamycin. Plasmid DNA from 3 kanamycin sensitive derivatives of BRL3745 and the one kanamycin sensitive derivative of 3746 was isolated and analyzed by agarose gel electrophoresis.

No plasmid DNA corresponding to the deletion 1 and deletion 3 plasmids was observed on the gel after curing. The BRL3745 derivatives cured of the deletion 1 plasmid were designated BRL3762. The BRL3746 derivative cured of the deletion 3 plasmid were designated BRL3763.

### EXAMPLE 15

### Competent Cells of BRL3762 and BRL3763.

Chemically competent cells of BRL3745, BRL3746, BRL3762 and BRL3763 were prepared according to the method of Hanahan (Hanahan D., 1983 J. Mol Biol 166,557) as modified according to U.S. Pat. No. 4,981,797. BRL3745 and BRL3746 were streaked on LB plates containing 20 μg/mL kanamycin and the plates were incubated at 28° C. for 20 hours. BRL3762(1), BRL3762(2), and BRL3763(1) were streaked on LB plates and the plates were incubated 28° C. for 20 hours. 5-6 colonies of each strain were picked into 1 mL SOB medium (D, Hanahan J. Mol Biol 166:557 1983). 0.9 mL of the cells were inoculated into 60 mL SOB medium in a 500 mL baffled shake flask. The flasks were placed in an 28° C. incubator 250 rpm. When the OD550 nm reached 0.25-0.33 the cells were harvested. 50 mL of cells of each strain were centrifuged (4° C.) and the cells were re-suspended in 4 mL cold CCMB80 buffer (D. Hanahan, J. Jessee and F. Bloom Methods in Enzymology 204:63 1991). The cells were allowed to sit on ice for 20 min. 220 μL were placed in NUNC vials and the cells were frozen in a dry ice ethanol bath. The cells were stored at −70° C.

### EXAMPLE 16

### Evaluation of Time to Ampicillin Resistant Colony.

One vial of competent cells (ATCC9637, BRL3734, BRL3745, BRL3746, BRL3762 and BRL3763) was thawed on ice for 20 min. 100 μL of cells were mixed in a cold Falcon 2059 tube with pUC19 (5 μL of 10 pg/μL=50 pg). The cells were allowed to sit on ice for 15 min. The cells were heat shocked at 42° C. for 45 seconds followed by a 2 minute incubation on ice. 0.9 mL of room temperature SOC was added to each tube and the tubes were shaken at 37° C. (250 rpm) for 30 minutes. Aliquots of the expression mix were plated on LB plates containing 100 μg/mL ampicillin and the plates were incubated at either 42° C. or 37° C. The time to the appearance of 1 mm colonies is shown in table 3. At 37° C., ampicillin resistant colonies of 1 mm size required 8.0 hours and there was no significant difference in time between strains containing both the 5.5 kb plasmid and the >50 kb plasmid (ATCC9637), strains containing only the >50 kb plasmid (BRL3734), strains containing the smaller kanamycin resistant plasmid (BRL3745 and 3746), or strains containing no plasmids (BRL3762 and 3763). At 42° C., colonies of 1 mm size required 7.3 hours for all strains tested. It was concluded that the presence or absence of plasmids in E. coli W does not significantly affect the time to appearance of colonies after transformation. In addition, the data in tables 3 and 4 indicate that incubation of the LB ampicillin plates at 42° C. results in the appearance of ampicillin resistant colonies approximately 0.5 hours faster than on plates incubated at 37° C.

TABLE 3
TIME IN HOURS TO AMPICILLIN RESISTANT COLONIES
AFTER TRANSFORMATION WITH PUC19 DNA.

Time to 1 mm
colony size

| STRAIN | 42° C. | 37° C. |
|---|---|---|
| ATCC 9637 | 7.3 | 8.0 |
| BRL3734 | 7.3 | 8.0 |
| BRL3745 | 7.3 | 8.0 |
| BRL3746 | 7.3 | 8.0 |
| BRL3762 (1) | 7.3 | 8.0 |
| BRL3762 (2) | 7.3 | 8.0 |
| BRL3763 | 7.3 | 8.0 |

TABLE 4
TIME IN HOURS TO AMPICILLIN RESISTANT COLONIES
AFTER TRANSFORMATION WITH PUC19 DNA

Time to 1 mm
colony size

| STRAIN | pUC19 | pBR322 |
|---|---|---|
| ATCC 9637 (W) recA⁺ | 8.0 | 8.5 |
| BRL3582(6) W recA⁻ | 10.25 | ND |
| MM294 recA⁺ | 10.25 | 10.25 |
| DH5α recA⁻ | 16.0 | 16.0 |

### EXAMPLE 17

### Comparison of wild-type E. coli W and E. coli K-12.

Competent cells of Escherichia coli strains ATCC9637 (W), BRL3582 (E. coli W endA⁻ sr1::Tn10 recA1398), and ATCC33625 (MM294) were prepared according to the method of Hanahan (Hanahan D., 1983 J. Mol Biol 166,557) as modified according to U.S. Pat. No. 4,981,797. The competent cells were prepared using CCMB80 buffer (Hanahan, D., Jessee, J., and Bloom, F. R., 1991, Methods in Enzymology 204,63). Max Efficiency DH5α competent cells were obtained from Life Technologies Inc.

The competent cells were thawed on ice for 20 minutes. 100 μL of the cells were transformed with 50 pg of pUC19 or 50 pg of pBR322 DNA. The cell-DNA mixture was placed on ice for 30 minutes and then heat shocked at 42° C. for 45 seconds. The tubes were then placed on ice for 2 minutes. 0.9 mL of SOC (Hanahan 1983) was added to each tube and the tubes were then shaken at 225 rpm for 1 hour at 37° C. Appropriate dilutions were spread on LB plates containing 100 μg/mL ampicillin and the plates were incubated at 37° C. The amount of time in hours to the appearance of 1 mm colonies was measured and is shown in Table 4. ATCC9637 yielded colonies in 8-8.5 hours compared to approximately 10 hours for ATCC33625, another recA⁺ strain. recA⁻ strains were also compared. BRL3582 yielded colonies in approximately 10 hours compared to 16 hours for DH5 cc.

### EXAMPLE 18

### Growth of Transformed Microorganisms at an Elevated Temperature.

Using the protocol described in the preceding example, the effects of growth an elevated temperature were analyzed. Incubating the transformed microorganisms on LB ampicillin plates at 42° C. resulted in the appearance of colonies from 0.5-1 hour faster compared to plates incubated at 37° C. Plating the cells on plates made from Circle Grow (Bio101) and containing ampicillin at 100 μg/mL resulted in the appearance of colonies from 0.5-1 hour faster compared to the appearance of colonies on LB plates containing ampicillin at 100 μg/mL. Thus, the use of elevated temperatures and/or enriched growth media may facilitate an increased growth rate of the microorganisms of the present invention.

### EXAMPLE 19

### Preparation of Derivatives of E. coli W Cured of Plasmids.

An isolate of E. coli W that has been cured of plasmid, such as BRL3762, BRL3763, BRL3756 or BRL3757, is used to construct derivatives having genotypes desirable for biotechnology applications. Using the P1 transduction technique described above, strains having one or more useful genetic alterations are prepared. Useful genetic alterations include: a recA⁻ genotype such as recA1/recA13 or recA deletions, a lacZ⁻ genotype that allows alpha complementation such as lacX74 lacZΔM 15 or other lacZ deletion, a protease deficient genotype such as Δlon and/or ompT⁻, an endonuclease minus genotype such as endA1, a genotype suitable for M13 phage infection by including the F′ episome, a restriction negative, modification positive genotype such as hsdR¹⁷(r_{K}⁻, m_{K}⁺), a restriction negative, modification negative genotype such as hsdS20(r_{B}⁻, m_{B}⁻), a methylase deficient genotype such as mcrA and/or mcrB and/or mrr, a genotype suitable for taking up large plasmids such as deoR, a genotype containing suppressor mutations such as supE and/or supF. Other suitable modifications are known to those skilled in the art and such modifications are considered to be within the scope of the present invention.

In a preferred embodiment, the rapid growing microorganisms of the present invention contains a modified lac operon that permits alpha complementation. In order to support alpha complementation, it was necessary to introduce a deletion into the N-terminal region of the genomic 1-galactosidase gene. First, a lacX74 mutation was introduced into BRL3756 and BRL3757 by P1 transduction with a lysate prepared on BRL3759 which contains the lacX74 mutation linked to a Tn10 insertion. Strains containing the lacX74 insertion are tetracycline resistant as a result of the Tn10 insertion. Strains were selected on tetracycline containing plates and the resultant strains were designated BRL3760 (derived from BRL3756) and BRL3761 (derived from BRL3757). The strains were cured of the Tn10 insertion by growth in the presence of fusaric acid and the resultant tetracycline sensitive strains containing the lacX74 mutation were designated BRL3766 and BRL769. These strains were made competent using the modified method of Hanahan as disclosed above and were then transformed with plasmid containing the alpha fragment of the β-galactosidase gene. The plasmid containing strains were transduced using a lysate prepared on and E. coli strains carrying the Φ80dlacZΔM15 deletion mutation linked to a Tn10 insertion in the trp gene. As a result of the insertion in the trp gene, strains carrying this mutation require tryptophan in the growth media. Tetracycline resistant strains were selected and were designated BRL3776 (derived from BRL3756 via BRL3760 and BRL3766) and BRL3778 (derived from BRL3757 via BRL3761 and BRL3769). These strains are lacX74 Φ80dlacZΔM15 trp⁻::Tn10. To restore the wild type trp gene, strains BRL3776 and BRL3778 were transduced with a P1 lysate prepared on E. coli DH5α and selected on minimal media minus tryptophan. The strains were spontaneously cured of the alpha fragment containing plasmid and the final alpha complementation strains BRL3781 (from BRL3776) and BRL3784 (from (BRL3778) were isolated. These strains are lacX74 Φ80dlacZΔM15. BRL3781 and BRL3784 were deposited at the Agricultural Research Service Culture Collection (NRRL, 1815 North University Street, Peoria, Ill., 61064) on Jun. 17, 1999. The deposits were made under the terms of the Budapest Treaty. BRL3781 has been given accession number NRRL No. B-30143 and BRL3784 has been given accession NRRL No. B-30144.

Those skilled in the art will appreciate that other modifications to the genome of the rapid growing microorganisms of the present invention are possible using the techniques described above. E. coli containing a desired mutation linked to a Tn10 insertion are readily available from sources well known to those skilled in the art. The desired mutation can be inserted into the genome of a rapid growing microorganism using P1 transduction and then the Tn10 can be cured by growth in the presence of fusaric acid.

In preferred embodiments, the rapid growing microorganisms of the present invention will carry an inducible T7 polymerase gene. In preferred embodiments, the T7 polymerase gene will be under the control of a salt inducible promoter as described by Bhandari, et al., J. Bacteriology, 179(13):4403-4406, 1997 which is specifically incorporated herein by reference. The T7 polymerase gene may be under the control of the salt inducible promoters of the proU locus. Alternatively, the T7 polymerase gene may be under the control of other salt inducible promoters. Other suitable inducible promoters include the lac promoter, the trp promoter, the tac promoter as well as any other inducible promoter known to those skilled in the art. The selection of the appropriate promoters and construction of strains carrying the T7 polymerase under the control of a given promoter are well within the abilities of those of ordinary skill in the art. Optionally, embodiments containing an inducible T7 polymerase gene may contain mutations in one or more protease genes and mutations in one or more ribonuclease genes. Such mutations may be inserted into the genome using the methods described above.

### EXAMPLE 20

### Evidence that E. coli strain W is lysogenic for bacteriophage Wphi

Plasmid pSPORT-1 isolated from E. coli strain W (BRL3763) remained uncut when treated with the restriction endonuclease NotI. Other restriction endonucleases, including EcoRI, BamHI and PstI, were able to digest the same plasmid to completion. Other plasmids containing NotI sites were digested to completion with NotI when isolated from strain DH10B, but were incompletely digested with NotI when isolated from strain W. These results suggested that DNA isolated from E. coli strain W had a site-specific modification at NotI sites rendering the DNA resistant to digestion with the NotI restriction endonuclease.

The site-specific modification at NotI sites was potentially due to methylase activity expressed by E. coli W. To test this possibility, a genomic DNA library was constructed from E. coli W in cosmid pCP13. The library was introduced into E. coli strain DH10B. DNA derived from clones coding for the E. coli W methylase should remain uncut when treated with NotI restriction endonuclease, while DNA derived from clones not containing the methylase will be digested. The cosmid library clones were isolated from DH10B transformants and then treated with NotI restriction endonuclease. The NotI-treated library clones were used to transform DH10B. Only uncut plasmids, i.e., those resistant to NotI enzyme, yielded colonies when transformed into DH10B. The cosmid clones containing NotI-resistant DNA were sub-cloned into a second plasmid. One cosmid clone containing NotI-resistant DNA was sequenced. The sequence of the clone revealed homology to the gene A of bacteriophage P2 and the 5mC methylases.

Bacteriophage capable of forming plaques on E. coli Cla were isolated from the supernatant of a culture of E. coli strain W. Experiments were performed to determine if the bacteriophage from E. coli W was identical to bacteriophage Wphi, a bacteriophage known to form plaques on E. coli Cla but not on DH5α. After plaque purification, the bacteriophage from stain W was used to lysogenize E. coli Cla. The lysogens were designated BRL3842 and BRL3843. Bacteriophage isolated from BRL3842 and BRL3843 did not form plaques on an authentic Wphi lysogen (C1920) but did form plaques on E. coli C1a. Likewise, bacteriophage isolated from C1920 did not form plaques on BRL3842 and BRL3843, but did form plaques on E. coli C1a. These results suggest that E. coli W is lysogenic for bacteriophage Wphi.

It was next determined whether Wphi encodes a methylase. The pSPORT-1 plasmid was transformed into competent cells of E. coli C1920 (the authentic Wphi lysogen), BRL3842 and BRL3843 (the two lysogens created from the bacteriophage of strain W) and E. coli C1a. Plasmid DNA was then isolated from the transformants and treated with NotI restriction endonuclease. Plasmid DNA prepared from E. coli C1a was digested to completion with NotI. Plasmid DNA prepared from C1920, however, could not be digested with NotI indicating that the Wphi bacteriophage DNA contained within C1920 encodes a methylase activity that prevents digestion of plasmid DNA with NotI. Furthermore, plasmid DNA prepared from BRL3842 and BRL3843 also could not be digested with NotI indicating the presence of the methylase in these strains and suggesting that the bacteriophage in BRL3842 and BRL3843 was Wphi.

### EXAMPLE 21

### Curing E. coli Strain W of Bacteriophage Wphi

The site of lysogenization for Wphi has been mapped at 88.6 min. on the E. coli chromosome. See Liu T., et al., J. Virol., 73:9816-9826 (1999). Experiments were conducted to displace the Wphi lysogen from E. coli strain W using recombination with a linked Tn10 transposon.

Competent cells of E. coli strain W were transformed with plasmid pCM301recA in order to make the strain phenotypically recA⁺. Plasmid pCM301recA is also temperature sensitive for replication; strains containing this plasmid must be grown on ampicillin plates at 30° C. A P1 vir lysate was obtained on strain CAG18477 (MG1655 metF159) containing Tn10 transposon zij501::Tn10 mapping at min 99.1. The P1 lysate was used to transduce the recA⁺ E. coli strain W described above, and transductants were selected for resistance to 20g/ml tetracycline at 30° C.

Twenty-four tetracycline resistant transductants, as well as the non-transduced recA⁺ E. coli strain W, were picked and grown in 1 ml LB broth at 30° C. to approximately 1×10⁹ cells/ml. The cells were centrifuged and the supernatants were treated with chloroform. The supernatants were spotted on a lawn of E. coli strain C117 (a P2 lysogen of E. coli C1a). A zone of clearing (indicating the presence of bacteriophage Wphi) was detected with the supernatant from the non-transduced recA⁺ E. coli strain W. The supernatants from two tetracycline resistant derivatives of recA⁺ E. coli strain W did not show any zone of clearing, indicating the absence of bacteriophage Wphi. These two strains, putatively cured of bacteriophage Wphi, were designated BRL3844-10 and BRL3844-15.

To determine whether BRL3844-10 and BRL3844-15 had been cured of bacteriophage Wphi, it was determined whether these strains exhibited the methylase activity detected in E. coli strain W. If BRL3844-10 and BRL3844-15 had been cured of Wphi, then plasmid pSPORT-1 transformed into and isolated from these strains should be capable of being digested by NotI enzyme due to the lack of methylase activity.

BRL3844-10 and BRL3844-15 were cured of plasmid pCM301recA by streaking the strains on LB plates at 42° C. and screening for ampicillin-sensitive colonies. The strains were designated as BRL3844-10A and BRL3844-15A. Competent cells of these cured strains were prepared and plasmid pSPORT-1 was transformed into the competent cells. Plasmid pSPORT-1 prepared from E. coli strain W could not be digested with NotI enzyme, indicating the presence of methylase activity. Plasmid pSPORT-1 prepared from BRL3844-10A and BRL3844-15A could be digested with NotI enzyme, indicating the absence of methylase activity. Therefore, curing E. coli strain W of the Wphi phage also eliminates the methylase activity associated with the Wphi phage.

Strains BRL3844-10A and BRL3844-15A were also tested by PCR to confirm that they did not contain bacteriophage Wphi nucleic acid. Primers were designed to anneal in the att, int, cox, P2, and methylase region of Wphi. PCR products of the expected sizes were generated from E. coli strain W; however, no PCR products were obtained from BRL3844-10A and BRL3844-15A, verifying that bacteriophage Wphi had been removed from these strains.

### EXAMPLE 22

### Detecting Bacteriophage Mu in E. coli Strain W

Bacteriophage Mu is a 43 kb phage that replicates by transposition. Since bacteriophage Mu is a transposon, it can potentially mutagenize any plasmid existing within the same cell and can infect most strains of E. coli. In addition, Mu is a lytic phage that has the potential to lyse E. coli cells. Bacteriophage Mu was detected in E. coli strain BRL3856 (ΔrecA1398, endA, fhuA, Φ80ΔlacM15, ΔlacX74, hsdR(r_{K}−m_{K}+)Mu⁺) by Southern blotting and by PCR analysis of chromosomal DNA using Mu-specific primers.

### EXAMPLE 23

### Deletion of Bacteriophage Mu from E. coli Strain W via Homologous Recombination Using Lambda Recombination Functions and Single Stranded Oligonucleotides

Oligonucleotides with short regions of homology (>30 bp) can be used to target genes in E. coli when lambda recombination functions (red α, β and γ proteins) are expressed prior to electroporation. See Ellis et al., Proc. Natl. Acad. Sci. USA 97:6742-6746 (2001). In order to delete the bacteriophage Mu genetic material from the genome of E. coli strain W using homologous recombination, a 153 bp single stranded oligonucleotide (MuKOFRT) was designed that carried 30 base pairs of sequences homologous to left and right ends of Mu flanking a single FRT site (5′-GGA CAT TGG ATT ATT CGG GAT CTG ATG GAT TAG TGT GTA GGC TGG AGC TGC TTC GAA GTT CCT ATA CTT TCT AGA GAA TAG GAA CTT CGG AAT AGG AAC TAA GGA GGA TAT TCA TAT GTT TGA AGC GCG AAA GCT AAA GTT TTC GCA TTT ATC-3′ (SEQ ID NO: 1)).

This single stranded targeting oligonucleotide, MuKOFRT, was used to delete the Mu phage genetic material from E. coli BRL3856 cells expressing lambda recombination functions α, β and γ from plasmid pKD46 (A plasmid that expresses lambda recombination functions from the arabinose inducible PBAD promoter and carries a temperature sensitive origin of replication. See Datsenko, K. A. and Wanner, B. L., Proc. Natl. Acad. Sci. USA 97:6640-6645 (2000)).

Plasmid pKD46 was introduced into chemically competent BRL3856, and plated at 30° C. on LB with ampicillin. A 2 ml overnight culture was prepared from a single colony in LB with ampicillin. One ml of overnight culture was used to inoculate 100 ml of SOB with ampicillin. Cells were grown at 30° C. to A₆₀₀=0.2 and 0.2% arabinose was added to induce lambda recombination functions. Cells were grown for 60 minutes and pelleted by centrifugation at 6000 RPM in an SS34 rotor. The cell pellet was resuspended 100 ml ice cold dH₂O and centrifuged at 6000 RPM to wash cells. This wash step was repeated, and the cell pellet was resuspended in 100 μl ice cold dH₂₀.

Oligonucleotide MuKOFRT (10 μg) carrying a single FRT recombination target and flanked by 30 bp of homology to the target sequence was mixed with 50 μl electrocompetent BRL3856/pKD46, and electroporated using a BTX electroporator set at 16 kV/cm, 25 μF and 200 ohm and a 0.1 cm cuvette. One ml SOC media was added, and cells were grown with agitation at 37° C. for 1 hour. Cells were diluted 1×10⁶ fold in SOC, and 10-100 μl of diluted cells were plated on LB plates. Cells were grown for 16 hours and 100 colonies were patched to fresh LB plates.

Colony PCR using primers specific for Mu (D5 and D6) and Platinum Taq Supermix was used to screen 45 colonies for the loss of Mu-specific sequences. Four colonies appeared to be missing Mu sequences by diagnostic PCR with primers D5 and D6 (see below for sequences of primers D5 and D6).

Southern blot analysis was performed to confirm the loss of Mu-specific sequences in the four ΔMu candidates. Chromosomal DNA from the AMu candidates was digested with EcoRI or SspI, resolved on a 0.8% agarose gel, transferred to a nylon membrane, and probed with ³²P-labeled Mu-specific PCR product. In addition, these four colonies were analyzed by PCR to further confirm loss of Mu-specific sequences. In the PCR analysis, Mu specific primer pairs E5/E6, E11/E12, D7/D8, and D9/D10 were used.

The sequences of primers D5, D6, E5, E6, E11, E12, D7, D8, D9 and D10 are as follows:

| | |
|---|---|
| D5:GAT CTG ATC GGA TTA GAT TTG GTG. | (SEQ ID NO:2) |
| D6:ATG ATG CTA GAT GCA TTA CCT GAA. | (SEQ ID NO:3) |
| E5:TTT GTA ACC GAC CTG TAT CAG AAA. | (SEQ ID NO:4) |
| E6:AGC ATC AAG AGG ATC CAT CAG. | (SEQ ID NO:5) |
| E11:GCA CAA TTA TTC AGA CAA AGC ACT. | (SEQ ID NO:6) |
| E12:ATC GTT ATC TCG TGA TAC CAC TCA. | (SEQ ID NO:7) |
| D7:GAT TCA GCA ACT GGA CGA GG. | (SEQ ID NO:8) |
| D8:AGT AAA AAC AGT CCT TTT GGA TCG. | (SEQ ID NO:9) |
| D9:GCA CTG CAA TTA ATA AAA CCA AAA. | (SEQ ID NO:10) |
| D10:ACT TAT GCT CCA TAA TTC TGA CCG. | (SEQ ID NO:11) |

Only one of the four candidates did not exhibit evidence of Mu genetic material by diagnostic PCR using all four primer pairs. This candidate, designated JDP674, was stocked as frozen. Strain JDP674 was deposited with NRRL, National Center for Agricultural Utilization Research, ARS, USDA, 1815 North University Street, Peoria, Ill. 61604, on Jan. 7, 2003, and has been assigned accession number NRRL B-30639.

Targeting oligonucleotide MuKOFRT was designed to introduce a single FRT site for further integration of FRT containing plasmids. For example, a plasmid with a conditional origin of replication, a promoter controlling the expression of a gene of interest, the gene of interest itself, and an antibiotic resistance gene can be integrated into JDP674 using a plasmid such as pCP20 (See Datsenko, K. A. and Wanner, B. L., Proc. Natl. Acad. Sci. USA 97:6640-6645 (2000)) to transiently supply FLP. Thus, the FRT site serves as a locus of integration for further strain construction.

### EXAMPLE 24

### Deletion of Bacteriophage Mu from Rapid Growing E. coli Using a Suicide Plasmid

A system for gene disruption that uses a suicide plasmid, pKAS32, with a conditional origin of replication, and the rpsL gene (streptomycin sensitivity) for selection against plasmid sequences has been described. See Skorupski, K. and Taylor, R. K., Gene 169:47-52 (1996). In order to delete a target on the genome, 600 base pair regions of homology are cloned upstream and downstream of a drug resistance cassette flanked by FRT sites, and introduced into pKAS32 (or into a plasmid that possesses the salient features of pKAS32) by restriction digestion/ligation. This method can be used to cure rapid growing E. coli of bacteriophage Mu genetic material.

To cure rapid growing E. coli of bacteriophage Mu genetic material using the system of Skorupski, the recipient strain, e.g., BRL3856, must first be made streptomycin resistant (Sm^{R}). The strain can be made Sm^{R} by, e.g., selecting for spontaneous mutants by plating >10⁶ cells on streptomycin plates and picking Sm^{R} colonies. The Sm^{R} derivative is then made recA⁺ by introducing a plasmid carrying recA (e.g., pHY100) by transformation, and selecting for tetracycline resistance on LB plates.

To design the targeting construct, the junction between the Mu ends and E. coli chromosome must be determined, e.g., by using the TOPO Walker® Kit (Invitrogen Corporation). Primers are then designed that introduce a restriction site for cloning of the cassette into pKAS32 (or similar plasmid) and are used to amplify about 600 base pairs from the Mu left end/E. coli chromosomal junction, and about 600 base pairs from the Mu right end/E. coli chromosomal junction. Primers are also designed to amplify the chloramphenicol resistance cassette flanked by FRT sites (FRT-CAT-FRT), e.g., from plasmid pKD3. See Datsenko, K. A. and Wanner, B. L., Proc. Natl. Acad. Sci. USA 97:6640-6645 (2000).

The PCR products will overlap allowing crossover PCR to be performed. The PCR products encoding the regions of homology flanking the target are mixed with the PCR product encoding the FRT sites surrounding the CAT gene, and the far left end and far right end flanking primers are used to amplify the 600 base pair regions of homology and drug resistance cassette. See Link et al., Journal of Bacteriology 179:6228-6237 (1997). The entire cassette is then amplified by PCR, and the FRT-CAT-FRT cassette PCR product is cut with a restriction enzyme encoded by the left end and right end flanking primers and ligated into similarly digested pKAS32. See Skorupski, K. and Taylor, R. K., Gene 169:47-52 (1996).

The resulting suicide plasmid is introduced into S17X pir (recA thi pro hsdR-M+ (RP4-2Tc::Mu Km::Tn7), see Skorupski, K. and Taylor, R. K., Gene 169:47-52 (1996)), and then is transferred into the Sm^{R} strain harboring a recA-carrying plasmid, e.g., BRL3858 Sm^{R}/pHY100, by conjugation. Transconjugates are selected on LB plates containing tetracycline and ampicillin. Ampicillin resistant colonies containing the integrated suicide plasmid are selected from each mating. Selection against the integrated plasmid by plating on LB streptomycin chloramphenicol plates is used to introduce the ΔMu::FRT-CAT-FRT mutation into the E. coli chromosome. Individual colonies are screened by PCR for the loss of Mu-specific sequences. The strain that is shown to lack Mu genetic material (e.g., BRL3856 SmR ΔMu::FRT-CAT-FRT) is then cured of the recA-carrying plasmid (e.g., PHY100) by growth in LB media without added antibiotics, and patching on LB tetracycline and LB chloramphenicol as a screen.

To remove the FRT-CAT-FRT cassette from the E. coli chromosome, a plasmid that expresses FLP recombinase, (e.g., pCP20), can be introduced into the strain that is shown to lack Mu genetic material (e.g., BRL3856 ΔMu::FRT-CAT-FRT). FLP expression can be induced by growth of transformants on LB plates at 42° C. Cells can be patched chloramphenicol plates to identify chloramphenicol sensitive colonies containing only a single FRT site. Colonies are then screened for the loss of Mu-specific sequences by PCR using Mu specific primers.

### EXAMPLE 25

### Deletion of Bacteriophage Mu from Rapid Growing E. coli Using Lambda Red-Mediated Recombination

Bacteriophage Mu can also be removed from rapid growing E. coli using lambda red-mediated recombination. See, Zhang et al., Nat. Genet. 20:123-128 (1998); Datsenko, K. A. and Wanner, B. L., Proc. Natl. Acad. Sci. USA 97:6640-6645 (2000). For example, a PCR product containing homology arms specific for Mu and priming sites specific for a CAT cassette flanked by FRT sites is designed. The template used to create the cassette used to replace Mu with FRT-CAT-FRT can be amplified from plasmid pKD3 using primers

| | |
|---|---|
| MuKO-R | |
| (5′-TGA AGC GGC GCA | (SEQ ID NO:12)) |
| CGA AAA ACG CGA AAG | |
| CGT TTC | ACG ATA AAT |
| GCG AAA ACT TTA GCT | |
| TTC GCG CTT CAA ACA | |
| TAT GAA TAT CCT CCT TAC-3′ | |
| and | |
| MuKO-L | |
| (5′-TGT ATT GAT TCA | (SEQ ID NO: 13)) |
| CTT GAA GTA CGA AAA | |
| AAA CCG GGA GGA CAT | |
| TGG ATT ATT CGG GAT | |
| CTG ATG GGA TTA GTG | |
| TGT AGG CTG GAG CTG CTT C-3′. | |

The cassette is amplified by PCR and introduced into electrocompetent rapid growing E. coli, e.g., BRL3856/pBADαβγ Amp cells, that are induced with 0.1% arabinose to express lambda recombination functions. Chloramphenicol resistant colonies are isolated, and grown overnight in LB without added chloramphenicol or ampicillin to enrich for strains that are cured of the pBADαβγ Amp plasmid. PCR with Mu specific primers and CAT specific primers can be used to confirm that the Mu genetic material is replaced with the FRT-CAT-FRT cassette.

To remove the FRT-CAT-FRT cassette from the E. coli chromosome, a plasmid that expresses FLP recombinase (e.g., pCP20), can be introduced into strains that are found to lack Mu genetic material, (e.g., BRL3856 ΔMu::FRT-CAT-FRT). FLP expression can be induced by growth of transformants on LB plates at 42° C. Cells can be patched chloramphenicol plates to identify chloramphenicol sensitive colonies containing only a single FRT site. Colonies are then screened for the loss of Mu-specific sequences by PCR using Mu specific primers.

### EXAMPLE 26

### Identification of Rapid Growing Microorganisms

Other microorganisms will be screened to identify rapid growing strains. Isolates to be screened are plated on an appropriate solid medium and grown to a defined colony size. The time to reach the defined colony size is compared to the time taken by an E. coli K or other strains described herein to reach the same colony size. The microorganisms to be screened include, but are not limited to, microorganisms such as those of the genera Escherichia sp. (particularly E. coli and, more specifically, E. coli strains B, C, W and K)), Klebsiella sp., Streptomyces sp., Streptocococcus sp., Shigella sp., Staphylococcus sp., Erwinia sp., Klebsiella sp., Bacillus sp. (particularly B. cereus, B. subtilis, and B. megaterium), Serratia sp., Pseudomonas sp. (particularly P. aeruginosa and P. syringae) and Salmonella sp. (particularly S. typhi or S. typhimurium). A plasmid conferring an antibiotic resistance is transformed into the microorganism to screened using the techniques described above. The transformed microorganisms are then plated on a solid medium containing antibiotic and then incubated at an appropriate temperature until colonies of a defined size are observed.

### EXAMPLE 27

### Cloning Using Rapid Growing Microorganisms.

The rapid growing microorganisms identified above may be used to clone DNA fragments. A population of recombinant vectors comprising a DNA insert having a desired sequence is constructed as described above. The vector may contain a DNA sequence coding for an antibiotic resistance gene and/or may contain one or more marker genes. The population of recombinant vectors is transformed into a rapid growing microorganism rendered competent by any conventional technique. For example, the microorganism is rendered competent by chemical means using the technique of Hanahan discussed above. Alternatively, the microorganism is made competent for electroporation by removing the growth media and placing the microorganism in a medium of low ionic strength. Any method of making the microorganism competent that allows the microorganism to take up exogenously applied DNA and, in particular, recombinant plasmids, is suitable for the practice of the instant invention.

Competent microorganisms are contacted with some or all of the population of recombinant vectors under conditions suitable to cause the uptake of the recombinant vectors into the competent microorganism. Suitable conditions may include a heat shock. For example, the mixture of cells and population of recombinant vectors are heated to 42° C. for 45 seconds. Alternatively, suitable conditions may include subjecting a mixture of microorganism and recombinant vector to an electric field.

After the recombinant vector is taken up by the microorganism, the microorganism is grown for a period of time sufficient to allow the expression of an antibiotic resistance gene. After any such period, the microorganism containing the recombinant vector is spread on plates containing the appropriate antibiotic and incubated until colonies are visible. In a preferred embodiment, the plates are incubated from about 4 hours to about 16 hours. In other preferred embodiments, the plates are incubated from about 4 hours to about 8 hours and in other preferred embodiments, the plates are incubated from about 4 hours to about six hours. In a preferred embodiment, the incubation step is performed at a temperature above 37° C. at which temperature the microorganism containing the recombinant plasmid grows more rapidly than it grows at 37° C. In another preferred embodiment, the incubation step is performed at 42° C.

After colonies become visible, some or all of the colonies are selected to be grown in liquid culture. The selection process may be by any conventional means. In a preferred embodiment, the microorganism and vector will permit alpha complementation and the selection is by blue/white screening on X-gal plates in the presence of IPTG. In other preferred embodiments, the selection is by detecting the presence or absence of a marker gene present on the vector. Suitable marker genes include, but are not limited to, the gene coding for luciferase, the gene coding for chloramphenicol acetyl transferase and the gene coding for β-glucuronidase.

The selected colonies are grown in liquid culture for a period of time sufficient to produce a quantity of recombinant microorganisms suitable for analysis. The recombinant vector is then isolated from the microorganisms. In a preferred embodiment, the period of growth in liquid culture is from about 2 hours to about 16 hours. In other preferred embodiments, the period of growth in liquid culture is from about 2 hours to about 8 hours and in other preferred embodiments, the period of growth in liquid culture is from about 2 hours to about 4 hours.

The recombinant vector is isolated by any conventional means. In a preferred embodiment, the recombinant vector is isolated by an alkaline lysis “mini-prep” technique. Optionally, the isolation may employ a column purification step. The isolated vector is analyzed by any conventional technique, for example, by agarose gel electrophoresis of the plasmid with or without prior digestion of the plasmid with one or more restriction enzymes. Other suitable techniques include sequencing of the plasmid. Techniques for determining the DNA sequence of a plasmid are well known to those skilled in the art.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. E. coli having a growth rate that is at least 5% greater than the growth rate of E. coli MM294, wherein said isolated E. coli do not contain any detectable levels of bacteriophage genetic material from at least one bacteriphage or in the alternative are resistant to infection by one or more bacteriophage types.

2. E. coli having a growth rate that is at least 5% greater than the growth rate of E. coli MM294, wherein said E. coli do not contain any detectable genetic material of bacteriophage Wphi.

3. E. coli having a growth rate that is at least 5% greater than the growth rate of E. coli MM294, wherein said E. coli do not contain any detectable genetic material of bacteriophage Mu.

4. The E. coli of claim 2, wherein said d E. coli additionally do not contain any detectable genetic material of one or more bacteriophage types selected from the group consisting of Mu, T1, T2, T3, T4, T5, T6 and T7.

5. The E. coli of claim 1, wherein said E. coli lack detectable levels of at least one endogenous plasmid.

6. The E. coli of claim 2, wherein said E. coli lack detectable levels of at least one endogenous plasmid.

7. The E. coli of claim 3, wherein said E. coli lack detectable levels of at least one endogenous plasmid.

8. The E. coli of claim 1, wherein said E. coli contain one or more genotype markers selected from the group consisting of: recA⁻, lacZ⁻, Δlon, ompT⁻, endA1, rnaE⁻, rnaI⁻, hsdR17(r_{K}⁻, m_{K}⁺), hsdS20(r_{B}⁻, m_{B}⁺), merA, mcrB, mrr, deoR, supE and supF.

9. The E. coli of claim 1, wherein said E. coli contain one or more genotype markers selected from the group consisting of: recA1, recA13, ΔrecA, lacX74, and lacZΔM15.

10. The E. coli of claim 1, wherein said E. coli contain an F′ episome or portions thereof.

11. The E. coli of claim 1, wherein said E. coli have a growth rate that is at least 25% greater than the growth rate of E. coli MM294.

12. The E. coli of claim 1, wherein said E. coli have a growth rate that is at least 50% greater than the growth rate of E. coli MM294.

13. The isolated E. coli of claim 1, wherein said E. coli have a growth rate that is at least 100% greater than the growth rate of E. coli MM294.

14. The E. coli of claim 1, wherein said E. coli are E. coli strain W or strain C.

15. A method of cloning comprising:
(a) obtaining competent E. coli;
(b) transforming said competent E. coli with at least one vector;
(c) selecting transformed E. coli containing said at least one vector; and
(d) culturing said transformed E. coli;
wherein said E. coli have a growth rate that is at least 5% greater than the growth rate of E. coli MM294, and wherein said E. coli do not contain any detectable levels of bacteriophage genetic material from at least one bacteriophage or in the alternative are resistant to infection by one or more bacteriophage types.

16. The method of claim 15 wherein said E. coli do not contain any detectable levels of genetic material of bacteriophage Wphi.

17. The method of claim 15 wherein said E. coli do not contain any detectable levels of genetic material of bacteriophage Mu.

18. The method of claim 15, wherein said E. coli lack detectable levels of at least one endogenous plasmid.

19. The method of claim 15, further comprising isolating said at least one vector from said transformed E. coli.

20. The method of claim 15, wherein the temperature at which said transformed E. coli are cultured is greater than 37° C.

21. The method of claim 20, wherein the temperature at which said transformed E. coli are cultured is about 42° C.

22. The method of claim 15, wherein said E. coli have a growth rate that is at least 25% greater than the growth rate of E. coli MM294.

23. The method of claim 15, wherein said E. coli have a growth rate that is at least 50% greater than the growth rate of E. coli MM294.

24. The method of claim 15, wherein said E. coli have a growth rate that is at least 100% greater than the growth rate of E. coli MM294.

25. The method of claim 15, wherein said E. coli are E. coli strain W or strain C.

26. The method of claim 15 wherein said E. coli is JDP674 or derivatives thereof.

27. A method of producing a protein or peptide, said method comprising:
(a) obtaining competent E. coli;
(b) transforming into said competent E. coli a vectorcontaining a gene encoding a protein or peptide; and
(c) culturing said transformed E. coli under conditions that cause said transformed E. coli to produce said protein or peptide;
wherein said E. coli have a growth rate that is at least 5% greater than the growth rate of E. coli MM294, and wherein said E. coli do not contain any detectable levels of bacteriophage genetic material from at least one bacterophage or in the alternative are resistant to infection by one or more bacteriophage types.

28. The method of claim 27 wherein said E. coli do not contain any detectable levels of genetic material of bacteriophage Wphi.

29. The method of claim 27 wherein said E. coli do not contain any detectable levels of genetic material of bacteriophage Mu.

30. The method of claim 27, wherein said E. coli lack any detectable levels of at least one endogenous plasmid.

31. The method of claim 27, wherein said E. coli have a growth rate that is at least 25% greater than the growth rate of E. coli MM294.

32. The method of claim 27, wherein said E. coli have a growth rate that is at least 50% greater than the growth rate of E. coli MM294.

33. The method of claim 27, wherein said E. coli have a growth rate that is at least 100% greater than the growth rate of E. coli MM294.

34. The method of claim 27, wherein said E. coli are strain W or strain C.

35. The method of claim 27 wherein said E. coli is JDP674 or derivatives thereof.

36. A method of transforming E. coli, said method comprising:
(a) obtaining competent E. coli;
(b) incubating said E. coli in the presence of one or more vectors under conditions which cause said one or more vectors to be taken up by said E. coli; and
(c) culturing said E. coli;
wherein said E. coli have a growth rate that is at least 5% greater than the growth rate of E. coli MM294, and wherein said E. coli do not contain any detectable levels of bacteriophage genetic material from at least one bacteriophage or in the alternative are resistant to infection by one or more bacteriophage types.

37. The method of claim 36 wherein said E. coli do not contain detectable levels of genetic material of bacteriophage Wphi.

38. The method of claim 36 wherein said E. coli do not contain detectable levels of genetic material of bacteriophage Mu.

39. The method of claim 36, wherein said E. coli lack detectable levels of at least one endogenous plasmid.

40. The method of claim 36, wherein said E. coli have a growth rate that is at least 25% greater than the growth rate of E. coli MM294.

41. The method of claim 36, wherein said E. coli have a growth rate that is at least 50% greater than the growth rate of E. coli MM294.

42. The method of claim 36, wherein said E. coli have a growth rate that is at least 100% greater than the growth rate of E. coli MM294.

43. The method of claim 36, wherein said E. coli are strain W or strain C.

44. The method of claim 36 wherein said E. coli is JDP674 or derivatives thereof.

45. A method of producing E. coli for cloning, said method comprising:
(a) obtaining E. coli having a growth rate that is at least 5% greater than the growth rate of E. coli MM294; and
(b) introducing into said E. coli a mutation that renders said E. coli resistant to infection by one or more bacteriophage types.

46. The method of claim 45, further comprising curing said E. coli of endogenous plasmids.

47. The method of claim 45, wherein said E. coli have a growth rate that is at least 25% greater than the growth rate of E. coli MM294.

48. The method of claim 45, wherein said E. coli have a growth rate that is at least 50% greater than the growth rate of E. coli MM294.

49. The method of claim 45, wherein said E. coli have a growth rate that is at least 100% greater than the growth rate of E. coli MM294.

50. The method of claim 45, wherein said E. coli are strain W or strain C.

51. The method of claim 45 wherein said E. coli is JDP674 or derivatives thereof.

52. A method of producing E. Coli for cloning, said method comprising:
(a) obtaining E. coli having a growth rate that is at least 5% greater than the growth rate of E. coli MM294, wherein said E. coli contain bacteriophage; and
(b) curing said E. coli of bacteriophage.

53. A method of producing E. coli for cloning, said method comprising:
(a) obtaining E. coli having a growth rate that is at least 5% greater than the growth rate of E. coli MM294, wherein said E. coli contain bacteriophage Wphi; and
(b) curing said E. coli of bacteriophage Wphi.

54. A method of producing E. coli for cloning, said method comprising:
(a) obtaining E. coli having a growth rate that is at least 5% greater than the growth rate of E. coli MM294, wherein said E. coli contain bacteriophage Mu; and
(b) curing said E. coli of bacteriophage Mu.

55. The method of claim 52, further comprising curing said E. coli of endogenous plasmids.

56. The method of claim 52, further comprising introducing into said E. coli a mutation that renders said E. coli resistant to infection by one or more bacteriophage types.

57. The method of claim 52, wherein said E. coli have a growth rate that is at least 25% greater than the growth rate of E. coli MM294.

58. The method of claim 52 wherein said E. coli have a growth rate that is at least 50% greater than the growth rate of E. coli MM294.

59. The method of claim 52, wherein said E. coli have a growth rate that is at least 100% greater than the growth rate of E. coli MM294.

60. The method of claim 52, wherein said E. coli are strain W or strain C.

61. The method of claim 52 wherein said E. coli is JDP674 or derivatives thereof.

62. A kit for cloning comprising a container containing E. coli having a growth rate that is at least 5% greater than the growth rate of E. coli MM294, wherein said E. coli do not contain detectable levels of bacteriophage genetic material from at least one bacteriophage or in the alternative are resistant to infection by one or more bacteriophages.

63. The kit of claim 62 wherein said E. coli is JDP674 or derivatives thereof.

64. A kit for cloning comprising a container containing E. coli having a growth rate that is at least 5% greater than the growth rate of E. coli MM294, wherein said E. coli do not contain detectable levels of genetic material of bacteriophage Wphi.

65. A kit for cloning comprising a container containing E. coli having a growth rate that is at least 5% greater than the growth rate of E. coli MM294, wherein said E. coli do not contain detectable levels of genetic material of bacteriophage Mu.

66. The kit of claim 62, wherein said E. coli lack detectable levels of at least one endogenous plasmid.

67. The kit of claim 62, further comprising one or more vector.

68. The kit of claim 66, further comprising at least one component selected from the group consisting of one or more restriction enzyme, one or more ligase enzyme, and one or more DNA polymerase.

69. The kit of claim 67, further comprising a container containing at least one recombination protein.

70. The kit of claim 62, wherein said E. coli contained within said kit are competent.

71. The kit of claim 70, wherein said E. coli contained within said kit are chemically competent.

72. The kit of claim 70, wherein said E. coli contained within said kit are electrocompetent.

73. The kit of claim 62, wherein said E. coli contained within said kit have a growth rate that is at least 25% greater than the growth rate of E. coli MM294.

74. The kit of claim 62, wherein said E. coli contained within said kit have a growth rate that is at least 50% greater than the growth rate of E. coli MM294.

75. The kit of claim 62, wherein said E. coli contained within said kit have a growth rate that is at least 100% greater than the growth rate of E. coli MM294.

76. The kit of claim 62, wherein said E. coli contained within said kit are strain W or strain C.

77. A composition comprising E. coli, wherein the E. coli of said composition have a growth rate that is at least 5% greater than the growth rate of E. coli MM294, and wherein said E. coli do not contain detectable levels of bacteriophage genetic material from at least one bacteriophage or in the alternative is resistant to infection by one or more bacteriophage types.

78. The composition of claim 77 wherein said E. coli is JDP674 or derivatives thereof.

79. A composition comprising E. coli, wherein the E. coli of said composition have a growth rate that is at least 5% greater than the growth rate of E. coli MM294, and wherein said E. coli do not contain detectable levels of genetic material of bacteriophage Wphi.

80. A composition comprising E. coli, wherein the E. coli of said composition have a growth rate that is at least 5% greater than the growth rate of E. coli MM294, and wherein said E. coli do not contain detectable levels of genetic material of bacteriophage Mu.

81. The composition of claim 77, wherein the E. coli of said composition lack detectable levels of at least one endogenous plasmid.

82. The composition of claim 77, further comprising a component selected from the group consisting of a glycerol solution and a competence buffer.

83. The composition of claim 77, further comprising at least one component selected from the group consisting of one or more DNA fragment, one or more ligase enzyme, one or more vector, one or more buffering salts, and one or more recombination protein.

84. The composition of claim 77, wherein the E. coli of said composition have a growth rate that is at least 25% greater than the growth rate of E. coli MM294.

85. The composition of claim 77, wherein the E. coli of said composition have a growth rate that is at least 50% greater than the growth rate of E. coli MM294.

86. The composition of claim 77, wherein the E. coli of said composition have a growth rate that is at least 100% greater than the growth rate of E. coli MM294.

87. The composition of claim 77, wherein the E. coli of said composition are E. coli strain W or strain C.

88. A method of making competent E. coli, said method comprising:
(a) obtaining E. coli having a growth rate that is at least 5% greater than the growth rate of E. coli MM294, wherein said E. coli do not contain detectable levels of bacteriophage genetic material from at least one bacteriophage or in the alternative are resistant to infection by one or more bacteriophage types; and
(b) treating said E. coli to make it competent.

89. The method of claim 88 wherein said E. coli do not contain detectable levels of genetic material of bacteriophage Wphi.

90. The method of claim 88 wherein said E. coli do not contain detectable levels of genetic material of bacteriophage Mu.

91. The method of claim 88, wherein said E. coli lack detectable lebvels of at least one endogenous plasmid.

92. The method of claim 88, wherein said E. coli have a growth rate that is at least 25% greater than the growth rate of E. coli MM294.

93. The method of claim 88, wherein said E. coli have a growth rate that is at least 50% greater than the growth rate of E. coli MM294.

94. The method of claim 88, wherein said E. coli have a growth rate that is at least 100% greater than the growth rate of E. coli MM294.

95. The method of claim 88, wherein said E. coli are E. coli strain W or strain C.

96. The method of claim 88 wherein said E. coli is JDP674 or derivatives thereof.

97. Competent E. coli having a growth rate that is at least 5% greater than the growth rate of E. coli MM294 wherein said E. coli do not contain detectable levels of bacteriophage genetic material of at least one bacteriophage or in the alternative are resistant to infection by one or more bacteriophage types.

98. A method for selecting for E. coli that contain a plasmid of interest, said method comprising:
(a) obtaining E. coli having a growth rate that is at least 5% greater than the growth rate of E. coli MM294, wherein said E. coli are unable to synthesize a cell membrane component thereby rendering said E. coli unable to grow in media lacking said cell membrane component;
(b) transforming said E. coli with a plasmid, wherein said plasmid encodes a gene product that restores the ability of said E. coli to grow in media lacking said cell membrane component; and
(c) culturing said transformed E. coli in medium lacking said cell membrane component.

99. The method of claim 98, wherein said cell membrane component is diaminopimelic acid.

100. The method of claim 99, wherein said E. coli are dap⁻.

101. The method of claim 99, wherein said gene product is diaminopimelic acid.

102. The method of claim 98, wherein said E. coli do not contain detectable levels of bacteriophage genetic material from at least one bacteriophage or in the alternative are resistant to one or more bacteriophage types.

103. The method of claim 98, wherein said E. do not contain detectable levels of genetic material of bacteriophage Wphi.

104. The method of claim 98, wherein said E. do not contain detectable levels of genetic material of bacteriophage Mu.

105. The method of claim 98 wherein said E. coli lack detectable levels of at least one endogenous plasmid.

106. The E. coli W derivative designated JDP674 and derivatives thereof.

107. The E. Coli W derivative designated JDP674.
